(19) United States
(12) Patent Application Publication
(10) Pub. No.: US 2021/0077659 A1
(43) Pub. Date: Mar. 18, 2021

(54) TISSUE-ADAPTIVE MATERIALS

(71) Applicant: The University of North Carolina at Chaptel Hill, Chapel Hill, NC (US)

(72) Inventors: Sergei Sheiko, Chapel Hill, NC (US); Mohammad VATANKHAH-VARNOSFADERANI, Coatesville, PA (US); Daixuan Zhang, Chapel Hill, NC (US); Erfan Dashtimoghadam, Chapel Hill, NC (US)

(21) Appl. No.: 16/962,477

(22) PCT Filed: Jan. 30, 2019
(86) PCT No.: PCT/US2019/015877

§ 371 Date: Jul. 15, 2020

Related U.S. Application Data
(60) Provisional application No. 62/623,878, filed on Jan. 30, 2018.

(51) Int. Cl. A61L 27/34; A61L 27/18; A61L 27/54

(52) U.S. Cl. CPC A61L 27/34; A61L 27/54; A61L 27/18

(57) ABSTRACT

The invention generally relates to polymer networks and methods of making and using same. Specifically, the disclosed polymer networks change mechanical properties upon insertion into a subject such as for example, a human. This abstract is intended as a scanning tool for purposes of searching in the particular art and is not intended to be limiting of the present invention.

### CROSS-REFERENCE TO RELATED APPLICATIONS

This application claims the benefit of U.S. Provisional Application No. 62/623,878, filed on Jan. 30, 2018, the contents of which is incorporated herein by reference in its entirety.

### STATEMENT REGARDING FEDERALLY SPONSORED RESEARCH

This invention was made with government support under grant no. DMR-1407645, awarded by the National Science Foundation (NSF). The government has certain rights in the invention.

### BACKGROUND

Mimicking biological tissues has always been a goal for material engineers because of its implications in biomedical engineering (e.g., implants, wearable electronics, and robotics). Current mimicking strategies are based on adding solvent, which allows tuning Young's modulus at small deformations; however, the addition of solvent fails to replicate the stress-strain behavior at large strains. Further, the constituting solvent may leak upon deformation, enhance swelling (i.e., shape change) in contact with bodily fluids, and also evaporate or freeze under variable environmental conditions. Finally, the existing materials for biomedical applications (and beyond) do not allow switching modulus by more than three orders of magnitude.

Despite the wide array of applications that could benefit from materials that closely mimic biological tissue, materials that match the mechanical properties of soft living tissue have yet to be realized. Therefore, there remains a need for materials that are mechanically matched to biological tissue. These needs and others are met by the present invention.

### SUMMARY

In accordance with the purpose(s) of the invention, as embodied and broadly described herein, the invention, in one aspect, relates to polymeric materials, methods of making same, and methods of treating a disorder comprising administering same.

Disclosed are polymer networks having an elastic modulus of at least about 10⁸ Pa at a temperature of less than about 75° F. and an elastic modulus of from about 10² Pa to about 10⁵ Pa at a temperature of greater than about 90° F.

Also disclosed are polymer networks comprising: (a) at least two polymer backbones; (b) a plurality of polymeric residues pendant from the polymer backbones, wherein the plurality of polymeric residues has a degree of polymerization of from about 1 to about 300, wherein the plurality of polymeric residues has a contour length of from about 1 nm to about 1 μm, wherein the plurality of polymeric residues has a softening transition temperature of from about −4° F. to about 140° F.; and (c) optionally, a side chain moiety pendant from the polymer backbones, wherein the side chain moiety either has a first binding functionality or is bonded to a reversible cross-link moiety, wherein the polymer network has a grafting density of from about 0.01 to about 1.

Also disclosed are polymer networks comprising the reaction product of: (a) a monomer selected from polyvalerolactone, polycarbonate, polycaprolactone methacrylate, polylactide methacrylate, polyglycolide methacrylate, polycaprolactone methacrylate, polycaprolactone acrylate, polylactide acrylate, polyethylene glycol, poly(2-ethyl-2-oxazoline), polyhydroxyalkanoate methacrylate, polyglycolide acrylate, and copolymers thereof; and (b) one or more of: (i) an irreversible cross-linker having two or more polymerizable functionalities selected from alkylene, alkene, acrylate, methacrylate, and epoxy; (ii) a reversible cross-linker having a second binding functionality, wherein the second binding functionality on one reversible cross-linker can bond to the second binding functionality on a second reversible cross-linker; and (iii) a reversible cross-linker having a pair of third binding functionalities; wherein the polymer network has a grafting density of from about 0.01 to about 1; and wherein the polymer network has a cross-linking density of from about 0.01 mole % to about 100 mole %.

Also disclosed are methods of making polymer networks.

Also disclosed are medical devices having a disclosed polymer network incorporated therein.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE DRAWINGS

The accompanying figures, which are incorporated in and constitute a part of this specification, illustrate several aspects and together with the description serve to explain the principles of the invention.

FIG. 1A shows a representative image of microneedles for non-invasive drug delivery on skin.

FIG. 1B shows representative scanning electron micrographs of microneedles fabricated with the disclosed materials (PCL).

FIG. 2 shows a representative image illustrating that the disclosed material is in a hard state at room temperature but becomes soft at body temperature.

FIG. 3A and FIG. 3B show representative diagrams illustrating hard-to-ultra soft transitions.

FIG. 4 shows a representative diagram illustrating that the disclosed materials have an unusually significant modulus drop between room temperature state and body temperature state.

FIG. 5 shows a 3D printed scaffold with the disclosed material (left) and a confocal image of cell-loaded scaffold (right).

FIG. 6A and FIG. 6B show representative diagrams illustrating that the mechanical properties can be costumed designed to fit various medical needs by polymer network architecture.

FIG. 7A-C show representative images illustrating a hard-to-super soft transition with dual property control.

FIG. 8 shows a representative ¹H NMR spectrum of oligomer of caprolactone produced using ring opening polymerization with a hydroxyl end group.

FIG. 9 shows a representative image illustrating dynamic network formation based on a Diels-Alder reaction between furan and maleimide functional groups on the backbone of the polymer chains and cross-linker, respectively.

FIG. 10 shows a representative diagram illustrating the temperature dependence of hard-to-soft elastomers.

FIG. 11A-D show representative diagrams illustrating thermo-mechanical properties of hard-to-soft bottlebrush polymers.

FIG. 12 shows representative images simulating microneedle insertion and drug release.

FIG. 13A and FIG. 13B show representative diagrams illustrating the control of release rate via polycaprolactone (PCL) networks.

FIG. 14A and FIG. 14B show representative diagrams illustrating release rate can be enhanced by adding polyethylene glycol side chains to PCL brush network strands that promote water uptake.

FIG. 15 shows a representative image illustrating dynamic network formation based on hydrogen bonding between ureidopyrimidinone functional groups on the backbone of the polymer chains.

Additional advantages of the invention will be set forth in part in the description that follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Further, the dates of publication provided herein may be different from the actual publication dates, which can require independent confirmation.

### A. Definitions

The present invention now will be described more fully hereinafter with reference to the accompanying drawings, in which illustrative aspects of the invention are shown. In the drawings, the relative sizes of regions or features may be exaggerated for clarity. This invention may, however, be embodied in many different forms and should not be construed as limited to the aspects set forth herein; rather, these aspects are provided so that this disclosure will be thorough and complete, and will fully convey the scope of the invention to those skilled in the art.

The disclosures of all patent references cited herein are hereby incorporated by reference to the extent they are consistent with the disclosure set forth herein. As used herein in the description of the invention and the appended claims, the singular forms “a,” “an,” and “the” are intended to include the plural forms as well, unless the context clearly indicates otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular aspects only and is not intended to be limiting of the invention. All publications, patent applications, patents and other references mentioned herein are incorporated by reference in their entirety. In case of a conflict in terminology, the present specification is controlling.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination. Moreover, the present invention also contemplates that in various aspects of the invention, any feature or combination of features set forth herein can be excluded or omitted. To illustrate, if the specification states that a complex comprises components A, B and C, it is specifically intended that any of A, B or C, or a combination thereof, can be omitted and disclaimed.

The term “about,” as used herein when referring to a measurable value, such as, for example, an amount or concentration and the like, is meant to encompass variations of ±20%, ±10%, ±5%, ±1%, ±0.5%, or even ±0.1% of the specified amount. A range provided herein for a measureable value may include any other range and/or individual value therein.

As used herein, “polymer network” refers to a polymer in which covalent or non-covalent (dynamic) cross-linking has occurred. Examples of polymer networks include, but are not limited to, polymer gels and elastomers.

As used herein, “softening transition temperature” refers to the temperature at which a substance undergoes a reversible change in state. For example, a substance can transition from a rubber-like state to a brittle state. Thus, in various aspects, a softening transition temperature can be a liquid transition temperature, a crystalline transition temperature, a melting transition temperature, or a glass transition temperature.

As used herein, “biocompatible” refers to materials that are not unduly reactive or harmful to a subject upon administration.

“Biodegradable” as used herein refers to the ability of a material to be broken down in vivo upon administration to a subject. For example, the materials may be dissolvable in skin tissue. See, e.g., Lee et al., “Dissolving Microneedles for Transdermal Drug Delivery,” Biomaterials 29(13):2113-2124, 2008. In various aspects, materials may be chosen to biodegrade at a predetermined rate, e.g., for controlled delivery of a therapeutic agent.

“Bioabsorbable” as used herein means capable of being absorbed into living tissue.

The term “therapeutic agent” is art-recognized and refers to any chemical moiety that is a biologically, physiologically, or pharmacologically active substance that acts locally or systemically in a subject. Examples of therapeutic agents, also referred to as “drugs,” are described in well-known literature references such as the Merck Index, the Physicians Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Various forms of a therapeutic agent may be used which are capable of being released from the subject composition into adjacent tissues or fluids upon administration to a subject. Examples include steroids and esters of steroids (e.g., estrogen, progesterone, testosterone, androsterone, cholesterol, norethindrone, digoxigenin, cholic acid, deoxycholic acid, and chenodeoxycholic acid), boron-containing compounds (e.g., carborane), chemotherapeutic nucleotides, drugs (e.g., antibiotics, antivirals, antifungals), enediynes (e.g., calicheamicins, esperamicins, dynemicin, neocarzino statin chromophore, and kedarcidin chromophore), heavy metal complexes (e.g., cisplatin), hormone antagonists (e.g., tamoxifen), non-specific (non-antibody) proteins (e.g., sugar oligomers), oligonucleotides (e.g., antisense oligonucleotides that bind to a target nucleic acid sequence (e.g., mRNA sequence)), peptides, proteins, antibodies, photodynamic agents (e.g., rhodamine 123), radionuclides (e.g., I-131, Re-186, Re-188, Y-90, Bi-212, At-211, Sr-89, Ho-166, Sm-153, Cu-67 and Cu-64), toxins (e.g., ricin), and transcription-based pharmaceuticals.

### B. Polymer Networks

In one aspect, disclosed are polymer networks having an elastic modulus of at least about 10⁸ Pa at a temperature of less than about 75° F. and an elastic modulus of from about 10² Pa to about 10⁵ Pa at a temperature of greater than about 90° F.

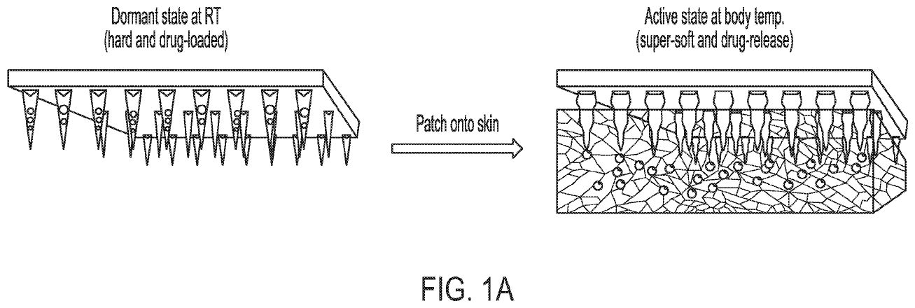

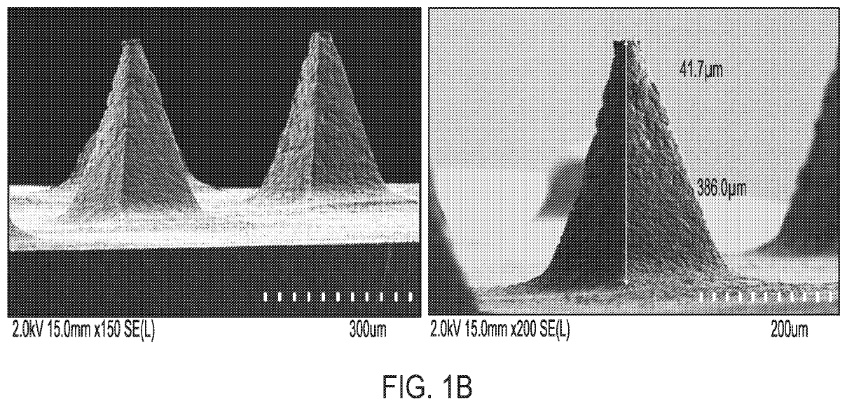

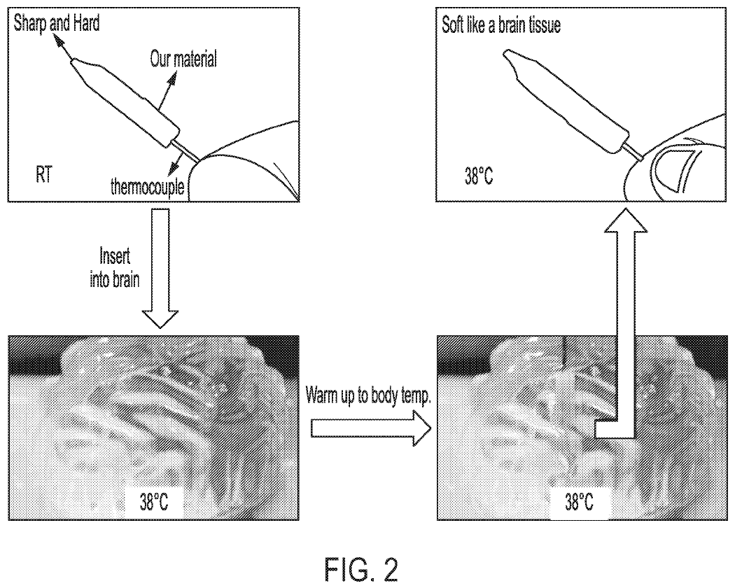

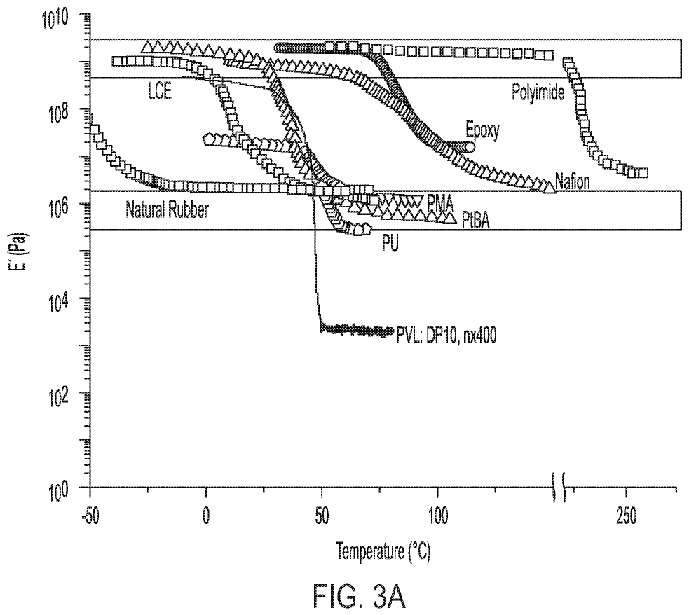

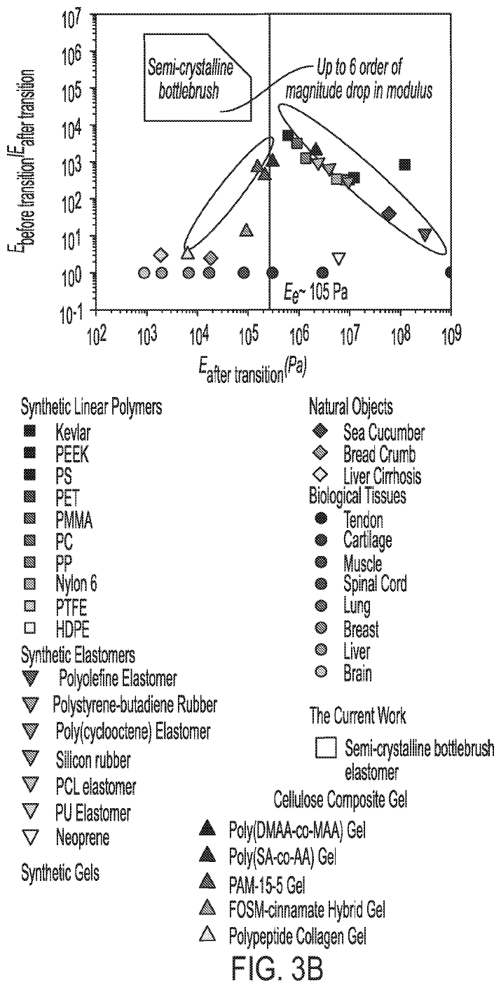

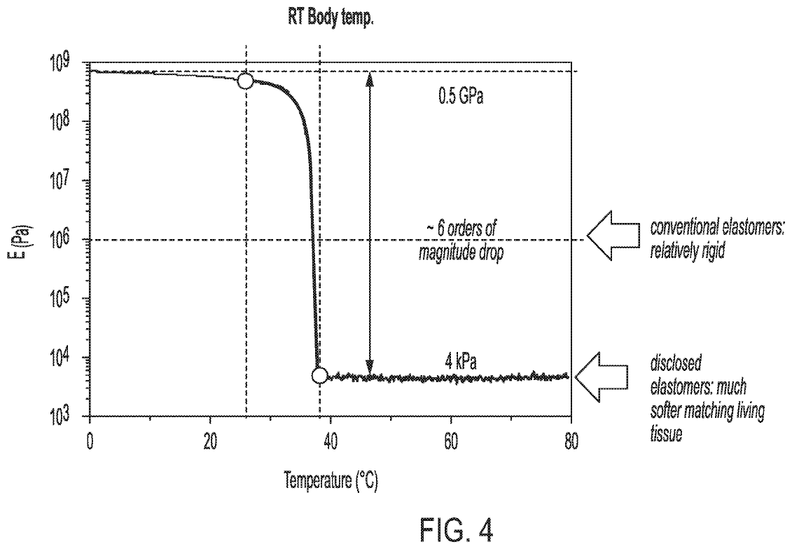

The disclosed polymer networks change mechanical properties upon insertion into a subject such as for example, a human (FIG. 1A). In various aspects, the disclosed polymer networks change mechanical properties upon physical contact with a subject. At ex vivo conditions, i.e., room temperature, these networks behave as rigid plastics that readily penetrate into a soft tissue, e.g., skin, brain, or liver (FIG. 2, FIG. 3A, and FIG. 3B). However, under in vivo conditions, i.e., subject body temperature, these networks become soft, matching the mechanical properties of the surrounding tissue (FIG. 3A, FIG. 3B, and FIG. 4). Optionally, these materials can be made moldable as a viscous liquid under high temperature, i.e., >100° C., to make objects such as, for example, implants, of complex shapes (FIG. 2).

Referring to FIG. 1A, at room temperature the needles remain hard and sharp, and loaded with drugs. Upon patching onto the skin, however, the needles warm up to body temperature, becoming as soft as skin tissue, and releasing drug at the same time. This could also be achieved on implant surfaces for easing inflammatory and rejection response.

Referring to FIG. 1B, scanning electron micrographs of microneedles fabricated with the disclosed materials (PCL) are shown.

Referring to FIG. 2, materials on the outside of a thermocouple, when at a hard state, enable easy penetration into soft tissues (e.g., brain). When warmed up to body temperature, however, the materials become as soft as the surrounding tissue (e.g., brain) and provide protection as a buffer layer between the soft brain tissue and the rigid thermocouple. This would find application in, for example, implants with encapsulation, from simple electrodes to complicated electronics.

Referring to FIG. 3A and FIG. 3B, the disclosed material is as soft as natural biological tissues, within a regime traditional elastomers can rarely reach (see FIG. 3B, left of the dashed line). Meanwhile, its modulus transition exceeds most known elastomeric system, enabling novel application such as, for example, self-penetrating implants.

Referring to FIG. 3A, many polymeric materials undergo a modulus decrease upon going through a thermal transitions (melting or glass-liquid). However, their modulus in the soft state cannot go below ˜10⁵-10⁶ Pa due to chain entanglements, which typically results in a limited modulus contrast of about 10³ or less. In contrast, bottlebrush elastomers with crystallizable side chains (e.g., PCL side chains) allow for much greater modulus drop from 1 GPa to 1 kPa, which corresponds to about 10⁵ to about 10⁶ of a modulus contrast.

Referring to FIG. 3B, the ratio of hard to soft moduli is plotted against modulus in the soft state. Most polymeric material has a similar modulus at hard state (˜1 GPa), so they are aligned along a line of −1 slope. Natural systems (circles and diamonds) do not have a reversible transition so their transitions are ignored. As shown, most biological tissues lay left of the 10⁵ mark. Gels (oval on left) can reach this area with limitations on transition due to their heterogenous nature. With respect to bottlebrush polymers, the general trend of polymers can be extended below the 10⁵ limit, yielding record high modulus drop (10⁵×) and tissue mimicking softness (10³-10⁵ Pa), as indicated by the polygon in the top left corner of the plot.

Referring to FIG. 4, the disclosed material has an unusually significant modulus drop between room temperature state and body temperature state. The entanglement bound (10⁵ Pa) applies as a lower bound for the modulus of the linear polymer melt. A bottlebrush defies such bound by not being able to entangle or by having very low entanglement. Thus, without wishing to be bound by theory, the upper bound allows piercing through tissue at room temperature while the lower bound matches the mechanical property of living tissue. This is ca. 3 orders of magnitude lower that of conventional elastomers such as, for example, poly(isoprene).

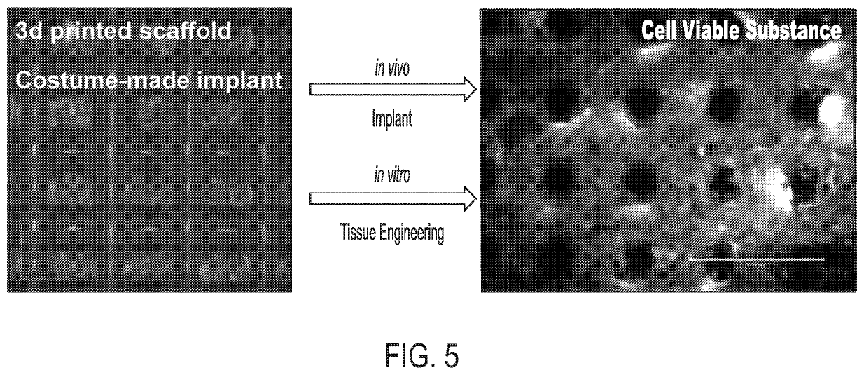

The material softening can also enhance diffusivity of smaller molecules, if present, and thus facilitate their release into the surrounding tissue (FIG. 5). Examples of smaller molecules include, but are not limited to, a drug, a contrast agent, and a lubricant. Areas for application of such diffusivity include, but are not limited to, drug delivery, tissue imaging, diagnostics, and joints lubrication.

Referring to FIG. 5, a 3D printed scaffold with the disclosed material (left) and confocal image of cell loaded scaffold (right) are shown. Without wishing to be bound by theory, the drastic change in modulus allows shaping and reshaping. In addition to customizable implants, the disclosed material is an ideal solution for in vitro tissue engineering scaffolds. The customizable modulus will provide a mechanical clue, which is known to trigger stem cell differentiation toward targeted tissue (e.g., chondrocytes and adipocytes), while the biocompatibility allows stem cell duplication. The confocal image shows great cell compatibility and adherence of the scaffold.

Further, due to the lack of solvent inside the disclosed polymer networks, the inserted objects demonstrate high acoustic contrast with respect to a surrounding tissue, despite having nearly identical mechanical properties. This feature is vital for areas such as ultrasound imaging and interrogation of the implants.

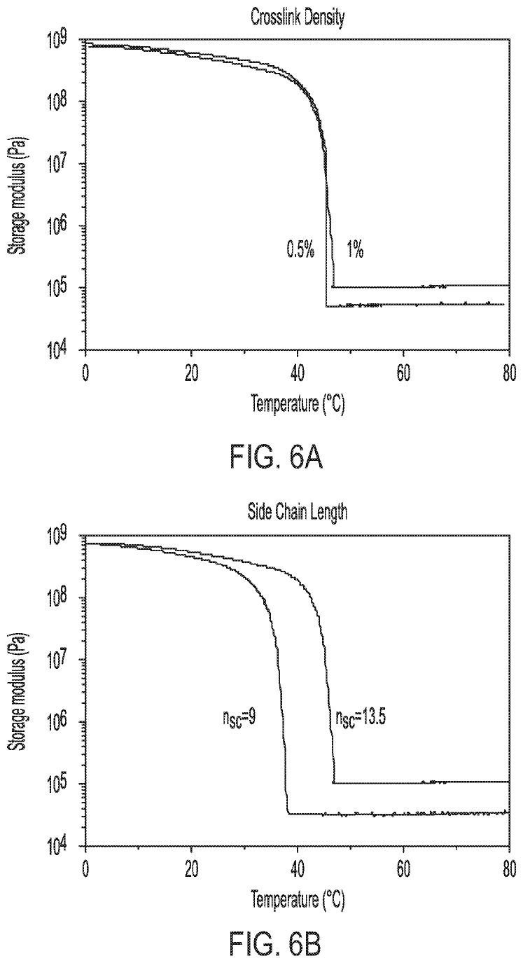

The in vivo softness of the disclosed polymer networks is ensured by crosslinking brush-like polymers using either permanent or reversible crosslinks or a combination thereof. The ex vivo hardness originates from crystallizable side chains that form a percolating scaffold of crystallites to support mechanical stress. Both the thermal and mechanical properties of these networks are controlled by the molecular architecture alone, without adding solvent and thus, changing the chemical composition (FIG. 6A and FIG. 6B). Without wishing to be bound by theory, the softening transition temperature is tuned by controlling the degree of polymerization of the side chain to ensure that it matches the body temperature. The in vivo softness or modulus is controlled by, for example, the length of the side chain, the grafting density, and the crosslinking density of the network. Without wishing to be bound by theory, independent synthetic control of these architectural parameters can allow for programmable design of materials with desired thermo-mechanical properties to satisfy both general application criteria and customized or personalized characteristics.

Referring to FIG. 6A and FIG. 6B, control of the modulus can be achieved to match specific organic tissue (FIG. 6A) and control of transition temperature to suit that of different in vivo conditions (FIG. 6B). None of these controls require a change in chemical composition, allowing for consistent biocompatibility.

The disclosed polymer networks have many advantages over existing technology. These advantages include minimally-invasive surgery for implant insertion, exact matching of the surrounding tissues mechanical properties, the matching does not require external stimulation, any adverse response due to mechanical mismatch with surrounding tissue is eliminated, enhanced physical comfort during carrying of implanted objects inside a body, biocompatible composition, non-toxic composition

In a further aspect, the polymer network is an elastomer.

In a further aspect, the polymer network is biocompatible. In a still further aspect, the polymer network is biodegradable. In a still further aspect, the polymer network is biocompatible and biodegradable.

In a further aspect, the polymer network has an elastic modulus of at least about 10⁹ Pa at a temperature of less than about 75° F. In a still further aspect, the polymer network has an elastic modulus of from about 3×10⁸ Pa at a temperature of less than about 75° F. In yet a further aspect, the polymer network has an elastic modulus of no more than about 8×10⁸ Pa at a temperature of less than about 75° F. In an even further aspect, the polymer network has an elastic modulus of from about 3×10⁸ Pa to about 8×10⁸ Pa at a temperature of less than about 75° F.

In a further aspect, the polymer network has a cross-linking density of from about 10⁻⁸ mol/cm³ to about 10⁻³ mol/cm³. In a still further aspect, the polymer network has a cross-linking density of from about 10⁻⁷ mol/cm³ to about 10⁻³ mol/cm³. In yet a further aspect, the polymer network has a cross-linking density of from about 10⁻⁶ mol/cm³ to about 10⁻³ mol/cm³. In an even further aspect, the polymer network has a cross-linking density of from about 10⁻⁵ mol/cm³ to about 10⁻³ mol/cm³. In a still further aspect, the polymer network has a cross-linking density of from about 10⁴ mol/cm³ to about 10⁻³ mol/cm³. In yet a further aspect, the polymer network has a cross-linking density of from about 10⁻⁸ mol/cm³ to about 10⁴ mol/cm³. In an even further aspect, the polymer network has a cross-linking density of from about 10⁻⁸ mol/cm³ to about 10⁻⁵ mol/cm³. In a still further aspect, the polymer network has a cross-linking density of from about 10⁻⁸ mol/cm³ to about 10⁻⁶ mol/cm³. In yet a further aspect, the polymer network has a cross-linking density of from about 10⁻⁸ mol/cm³ to about 10⁻⁷ mol/cm³. In an even further aspect, the polymer network has a cross-linking density of from about 10⁻⁷ mol/cm³ to about 10⁴ mol/cm³. In a still further aspect, the polymer network has a cross-linking density of from about 10⁻⁶ mol/cm³ to about 10⁻⁵ mol/cm³.

1. Structure

In one aspect, the polymer network comprises: (a) at least two polymer backbones; (b) a plurality of polymeric residues pendant from the polymer backbones, wherein the plurality of polymeric residues has a degree of polymerization of from about 1 to about 300, wherein the plurality of polymeric residues has a contour length of from about 1 nm to about 1 μm, wherein the plurality of polymeric residues has a softening transition temperature of from about −4° F. to about 140° F.; and (c) optionally, a side chain moiety pendant from the polymer backbones, wherein the side chain moiety either has a first binding functionality or is bonded to a reversible cross-link moiety, wherein the polymer network has a grafting density of from about 0.01 to about 1.

In a further aspect, each polymer backbone is biocompatible. In a still further aspect, each polymer backbone is biodegradable. In a still further aspect, each polymer backbone is biocompatible and biodegradable.

In a further aspect, the polymer backbone is a polyester backbone, a polyacrylate backbone, or a methacrylate backbone.

In a further aspect, the plurality of polymeric residues are biocompatible. In a still further aspect, the plurality of polymeric residues are biodegradable. In a still further aspect, the plurality of polymeric residues are biocompatible and biodegradable.

In a further aspect, the plurality of polymeric residues has a degree of polymerization of from about 1 to about 250. In a still further aspect, the plurality of polymeric residues has a degree of polymerization of from about 1 to about 200. In yet a further aspect, the plurality of polymeric residues has a degree of polymerization of from about 1 to about 100. In an even further aspect, the plurality of polymeric residues has a degree of polymerization of from about 1 to about 50. In a still further aspect, the plurality of polymeric residues has a degree of polymerization of from about 50 to about 300. In yet a further aspect, the plurality of polymeric residues has a degree of polymerization of from about 100 to about 300. In an even further aspect, the plurality of polymeric residues has a degree of polymerization of from about 150 to about 300. In a still further aspect, the plurality of polymeric residues has a degree of polymerization of from about 200 to about 300. In yet a further aspect, the plurality of polymeric residues has a degree of polymerization of from about 250 to about 300.

In a further aspect, the plurality of polymeric residues has a contour length of from about 0.5 nm to about 2 μM. In a still further aspect, the plurality of polymeric residues has a contour length of from about 0.5 nm to about 1.5 μM. In yet a further aspect, the plurality of polymeric residues has a contour length of from about 0.5 nm to about 1 μM. In an even further aspect, the plurality of polymeric residues has a contour length of from about 0.5 nm to about 0.5 μM. In a still further aspect, the plurality of polymeric residues has a contour length of from about 0.5 nm to about 0.1 μM. In yet a further aspect, the plurality of polymeric residues has a contour length of from about 1 nm to about 2 μM. In an even further aspect, the plurality of polymeric residues has a contour length of from about 5 nm to about 2 μM. In a still further aspect, the plurality of polymeric residues has a contour length of from about 10 nm to about 2 μM. In yet a further aspect, the plurality of polymeric residues has a contour length of from about 100 nm to about 2 μM. In an even further aspect, the plurality of polymeric residues has a contour length of from about 1 nm to about 1 μM.

In a further aspect, the plurality of polymeric residues has a softening transition temperature of from about −4° F. to about 100° F. In a still further aspect, −4° F. to about 80° F. In yet a further aspect, −4° F. to about 50° F. In yet a further aspect, −4° F. to about 10° F. In an even further aspect, 0° F. to about 140° F. In a still further aspect, 10° F. to about 140° F. In yet a further aspect, 50° F. to about 140° F. In an even further aspect, 80° F. to about 140° F.

In a further aspect, the softening transition temperature is a melting transition temperature. In a still further aspect, the softening transition temperature is a liquid transition temperature.

In a further aspect, each polymeric residue is a polyester residue, a polyacrylate residue, or a polymethacrylate residue. In a still further aspect, each polymeric residue is selected from a polycaprolactone residue, a polylactide residue, a polyether residue, a polycarbonate residue, a polyvalerolactone residue, and a poly(lactic-co-glycolic) acid residue. In yet a further aspect, each polymeric residue is a polycaprolactone residue. In an even further aspect, each polymeric residue is a polyvalerolactone residue.

In a further aspect, each polymeric residue has a structure:

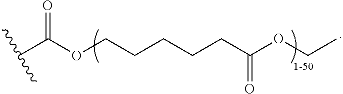

In a further aspect, each polymeric residue has a structure:

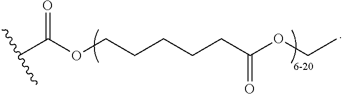

In a further aspect, each polymeric residue has a structure:

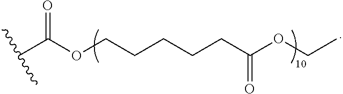

In a further aspect, each polymeric residue has a structure:

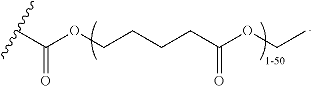

In a further aspect, each polymeric residue has a structure:

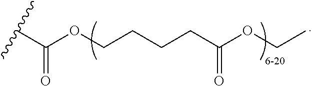

In a further aspect, each polymeric residue has a structure:

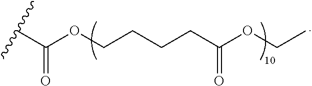

In a further aspect, the side chain moiety is present. In a still further aspect, the side chain moiety is absent.

In a further aspect, the side chain moiety is a residue having a structure selected from:

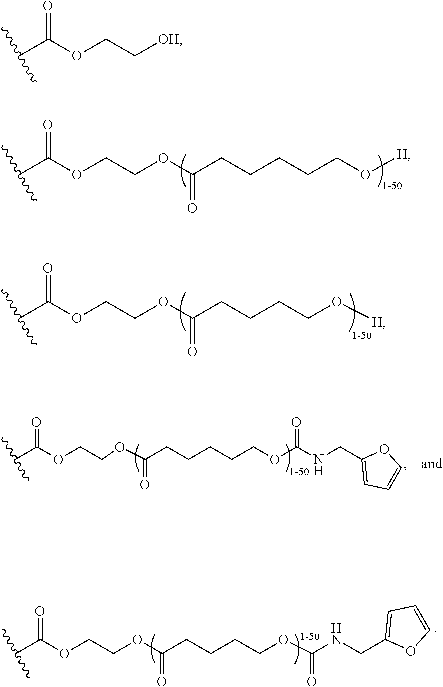

In a further aspect, the side chain moiety is a residue having a structure selected from:

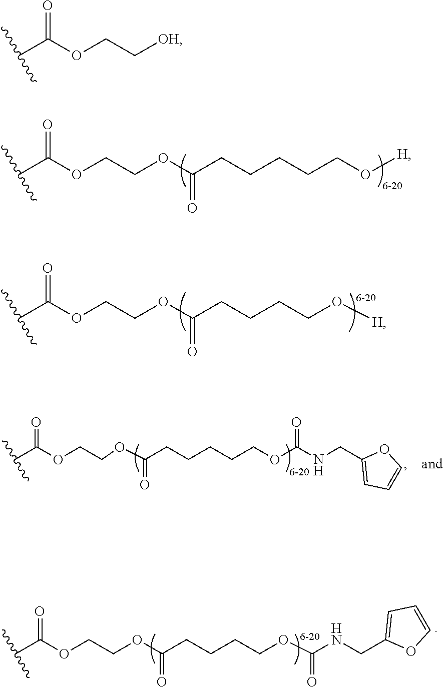

In a further aspect, the side chain moiety is a residue having a structure selected from:

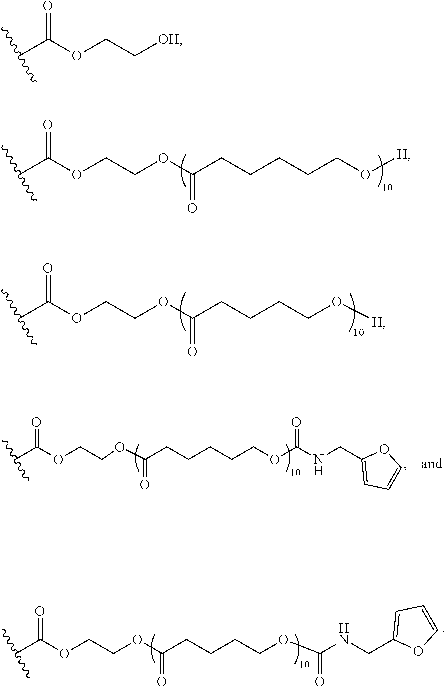

In a further aspect, the side chain moiety has a first binding functionality. Examples of first binding functionalities include, but are not limited to, hydroxyl moieties, amino moieties, carboxylic acid moieties, amide moieties, urea moieties, and furan moieties. In a still further aspect, the first binding functionality is selected from hydroxyl, amine, and furan. In yet a further aspect, the first binding functionality is a hydroxyl. In yet a further aspect, the first binding functionality is a furan.

In a further aspect, the side chain moiety is bonded to a reversible cross-link moiety. In a still further aspect, bonded is via a hydrogen bond, a Diels alder reaction, a dithiol bond, a metal-ligand bond, pi-pi stacking, or hydrophobic-hydrophobic interactions.

In a further aspect, the polymer network has a grafting density of from about 0.01 to 100. In a still further aspect, the polymer network has a grafting density of from about 0.01 to 75. In yet a further aspect, the polymer network has a grafting density of from about 0.01 to 50. In an even further aspect, the polymer network has a grafting density of from about 0.01 to 25. In a still further aspect, the polymer network has a grafting density of from about 0.01 to 10. In yet a further aspect, the polymer network has a grafting density of from about 0.01 to 1. In an even further aspect, the polymer network has a grafting density of from about 1 to 100. In a still further aspect, the polymer network has a grafting density of from about 10 to 100. In yet a further aspect, the polymer network has a grafting density of from about 25 to 100. In an even further aspect, the polymer network has a grafting density of from about 50 to 100. In a still further aspect, the polymer network has a grafting density of from about 75 to 100.

In a further aspect, the polymer network further comprises one or more of: (a) an irreversible cross-link moiety covalently bonded to the two polymer backbones; (b) two reversible cross-link moieties having a first end and a second end, wherein each first end is covalently bonded to one of the two polymer backbones, and wherein each second end is bonded to each other; and (c) a reversible cross-link moiety having a first end and a second end, wherein the first end is bonded to one side chain moiety, and wherein the second end is bonded to a different side chain moiety.

In a further aspect, one or more of the irreversible cross-link moiety, two reversible cross-link moieties, and reversible cross-link moiety is biocompatible. In a still further aspect, one or more of the irreversible cross-link moiety, two reversible cross-link moieties, and reversible cross-link moiety is biodegradable.

In a further aspect, the irreversible cross-link moiety is biocompatible. In a still further aspect, the irreversible cross-link moiety is biodegradable. In a still further aspect, the irreversible cross-link moiety is biocompatible and biodegradable.

In a further aspect, the two reversible cross-link moieties are biocompatible. In a still further aspect, the two reversible cross-link moieties are biodegradable. In a still further aspect, the two reversible cross-link moieties are biocompatible and biodegradable.

In a further aspect, the reversible cross-link moiety is biocompatible. In a still further aspect, the reversible cross-link moiety is biodegradable. In a still further aspect, the reversible cross-link moiety is biocompatible and biodegradable.

In a further aspect, the polymer network comprises the irreversible cross-link moiety covalently bonded to the two polymer backbones. Examples of irreversible cross-link moieties include, but are not limited to, polycaprolactone residues, polyvalerolactone residues, polylactide residues, poly(lactic-co-glycolic) residues, polyether residues, and polycarbonate residues. In a still further aspect, the irreversible cross-link moiety is a residue having a structure:

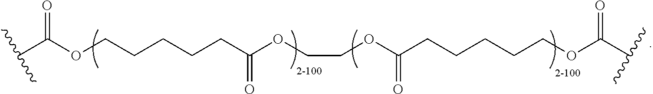

In yet a further aspect, the irreversible cross-link moiety is a residue having a structure:

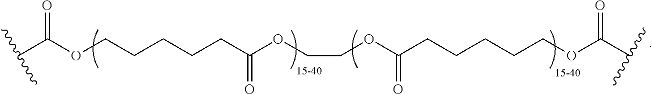

In an even further aspect, the irreversible cross-link moiety is a residue having a structure:

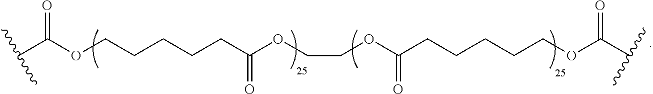

In a still further aspect, the irreversible cross-link moiety is a residue having a structure:

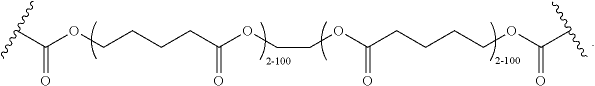

In yet a further aspect, the irreversible cross-link moiety is a residue having a structure:

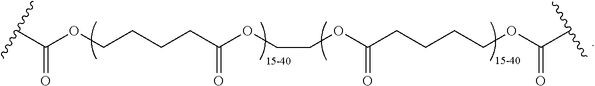

In an even further aspect, the irreversible cross-link moiety is a residue having a structure:

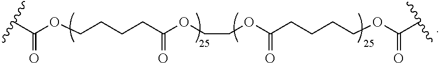

In a further aspect, the polymer network comprises two reversible cross-link moieties having a first end and a second end. Examples of reversible cross-link moieties include, but are not limited to, terpyridine residues, ureidopyrimidinone residues, maleimide residues, catechol residues, thiol residues, furfuryl residues, azide residues, alkyne residues, alkene residues, amine residues, aldehyde residues, isocyanate residues, and hydroxyl residues. In a still further aspect, the reversible cross-link moiety is an ureidopyrimidinone residue. In yet a further aspect, the ureidopyrimidinone residue has a structure:

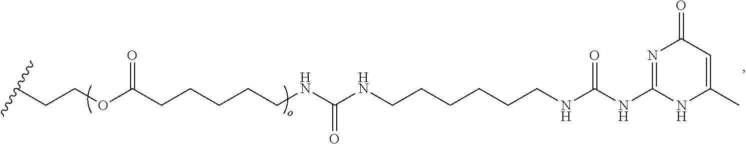

wherein each occurrence of o is independently an integer selected from 2 to 50. In an even further aspect, each occurrence of o is independently an integer selected from 6-20. In an even further aspect, the reversible cross-link moiety is a residue that can participate in a hydrogen bond.

In a further aspect, each second end is non-covalently or dynamically bonded to each other. In a still further aspect, each second end is bonded to each other via a hydrogen bond, a Diels alder reaction, a dithiols bond, a metal-ligand bond, pi-pi stacking, or hydrophobic-hydrophobic interactions.

In a further aspect, the polymer network comprises the reversible cross-link moiety having a first end and a second end. Examples of reversible cross-link moieties include, but are not limited to, terpyridine residues, ureidopyrimidinone residues, maleimide residues, catechol residues, thiol residues, and furfuryl residues. In a still further aspect, the reversible cross-link moiety is a dimaleimide residue. In yet a further aspect, the dimaleimide residue has a structure:

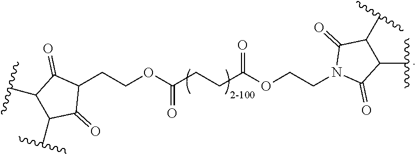

In an even further aspect, the dimaleimide residue has a structure:

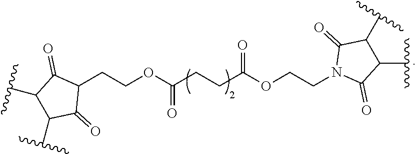

In a still further aspect, the reversible cross-link moiety is a residue that can participate in a hydrogen bond.

In a further aspect, the first end is covalently bonded to one side chain moiety and wherein the second end is covalently bonded to a different side chain moiety.

In a further aspect, the second end is non-covalently or dynamically bonded to each other. In a still further aspect, the second end is bonded to each other via a hydrogen bond, a Diels alder reaction, a dithiols bond, a metal-ligand bond, pi-pi stacking, or hydrophobic-hydrophobic interactions.

In a further aspect, the polymer network has a cross-linking density of from about 10⁻⁸ mol/cm³ to about 10⁻³ mol/cm³. In a still further aspect, the polymer network has a cross-linking density of from about 10⁻⁷ mol/cm³ to about 10⁻³ mol/cm³. In yet a further aspect, the polymer network has a cross-linking density of from about 10⁻⁶ mol/cm³ to about 10⁻³ mol/cm³. In an even further aspect, the polymer network has a cross-linking density of from about 10⁻⁵ mol/cm³ to about 10⁻³ mol/cm³. In a still further aspect, the polymer network has a cross-linking density of from about 10⁴ mol/cm³ to about 10⁻³ mol/cm³. In yet a further aspect, the polymer network has a cross-linking density of from about 10⁻⁸ mol/cm³ to about 10⁴ mol/cm³. In an even further aspect, the polymer network has a cross-linking density of from about 10⁻⁸ mol/cm³ to about 10⁻⁵ mol/cm³. In a still further aspect, the polymer network has a cross-linking density of from about 10⁻⁸ mol/cm³ to about 10⁻⁶ mol/cm³. In yet a further aspect, the polymer network has a cross-linking density of from about 10⁻⁸ mol/cm³ to about 10⁻⁷ mol/cm³. In an even further aspect, the polymer network has a cross-linking density of from about 10⁻⁷ mol/cm³ to about 10⁻⁴ mol/cm³. In a still further aspect, the polymer network has a cross-linking density of from about 10⁻⁶ mol/cm³ to about 10⁻⁵ mol/cm³.

### C. Methods of Making Polymer Networks

In one aspect, disclosed are methods of making a disclosed polymer network.

Thus, in one aspect, the polymeric network comprises the reaction product of: (a) a monomer selected from polyvalerolactone, polycarbonate, polycaprolactone methacrylate, polylactide methacrylate, polyglycolide methacrylate, polycaprolactone methacrylate, polycaprolactone acrylate, polylactide acrylate, polyethylene glycol, poly(2-ethyl-2-oxazoline), polyhydroxyalkanoate methacrylate, polyglycolide acrylate, and copolymers thereof; and (b) one or more of: (i) an irreversible cross-linker having two or more polymerizable functionalities selected from alkylene, alkene, acrylate, methacrylate, and epoxy; (ii) a reversible cross-linker having a second binding functionality, wherein the second binding functionality on one reversible cross-linker can bond to the second binding functionality on a second reversible cross-linker; and (iii) a reversible cross-linker having a pair of third binding functionalities; wherein the polymer network has a grafting density of from about 0.01 to about 1; and wherein the polymer network has a cross-linking density of from about 0.01 mole % to about 100 mole %. In a further aspect, the monomer has an active site selected from acrylate, methacrylate, and norbornene.

In one aspect, the polymeric network comprises the reaction product of: (a) a polymer having a residue of polyvalerolactone, polylactide, poly(lactic-co-glycolic), polyether, polycarbonate acrylate, polycarbonate methacrylate, polycarbonate norbornene, polycaprolactone methacrylate, polylactide methacrylate, polyglycolide methacrylate, polycaprolactone methacrylate, polycaprolactone acrylate, polyhydroxyethyl acrylate, polylactide acrylate, ethylene glycol methacrylate, ethylene glycol acrylate, poly(2-ethyl-2-oxazoline), or polyglycolide acrylate; and (b) one or more of: (i) an irreversible cross-linker having two or more polymerizable functionalities selected from alkylene, alkene, acrylate, methacrylate, and epoxy; (ii) a reversible cross-linker having a second binding functionality, wherein the second binding functionality on one reversible cross-linker can bond to the second binding functionality on a second reversible cross-linker; and (iii) a reversible cross-linker having a pair of third binding functionalities; wherein the polymer network has a grafting density of from about 0.01 to about 1; and wherein the polymer network has a cross-linking density of from about 0.01 mole % to about 100 mole %. In a further aspect, the polymer has a residue of polyvalerolactone, polylactide, poly(lactic-co-glycolic), polyether and polycarbonate. In a still further aspect, the polymer has an active site selected from acrylate, methacrylate, and norbornene.

In a further aspect, the monomer is biocompatible. In a still further aspect, each the monomer is biodegradable. In a still further aspect, the monomer is biocompatible and biodegradable.

In various aspects, the polymer network is prepared by one-pot graft-through polymerization. This can be done, for example, by reacting monomers with photo-activated crosslinkers. Without wishing to be bound by theory, both the monomer and the cross-linker can be synthesized by ring opening polymerization (ROP) in the presence of an initiator, e.g., ethanol or ethylene glycol, followed by reaction with, for example, methacryloyl chloride.

Thus, in a further aspect, the monomer is selected from polycaprolactone methacrylate, polylactide methacrylate, polyglycolide methacrylate, polyhydroxyethyl acrylate, polylactide acrylate, and polyglycolide acrylate. In a still further respect, the monomer is polycaprolactone methacrylate. In yet a further aspect, the monomer has a structure:

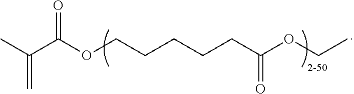

In an even further aspect, the monomer has a structure:

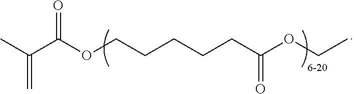

In a still further aspect, the monomer has a structure:

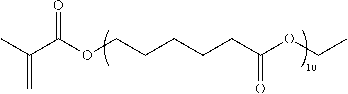

In a further aspect, the monomer is reacted with the irreversible cross-linker. Examples of irreversible cross-linkers include, but are not limited to, polycaprolactone, polylactide, polyglycolide, a co-polymer of lactide, a co-polymer of glycolide, poly(ethylene glycol), polytetramethylene oxide, and polyoxazoline. In a still further aspect, the irreversible cross-linker has a structure:

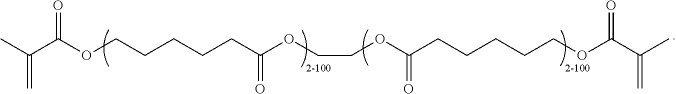

In yet a further aspect, the irreversible cross-linker has a structure:

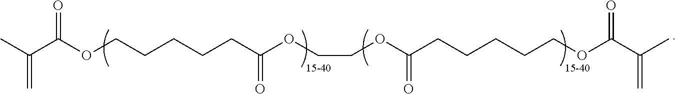

In an even further aspect, the irreversible cross-linker has a structure:

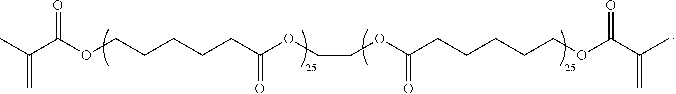

In various aspects, the polymer network is prepared by first synthesizing the polymer backbone via free radical polymerization (FRP), atom transfer radical polymerization (ATRP), SARA ATRP, or reversible addition-fragmentation chain-transfer polymerization (RAFT), and then grafting from a functionality on the polymer backbone. Prior to grafting from, the monomer can be further functionalized using, for example, ring opening polymerization of caprolactone (CL), lactic acid (LA), glycolic acid (GA), valerolactone, a polylactide residue, and a lactic-co-glycolic residue, to the specific degree of polymerization to tune the transition temperature of the brush-like polymer.

Thus, in various aspects, the monomer has a structure:

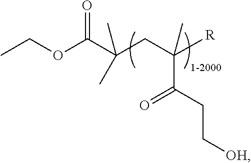

wherein R is Br if the backbone is synthesized through ATRP, H if synthesized through FRP, and a RAFT moiety if synthesized through RAFT.

In a further aspect, the monomer has a structure:

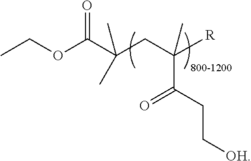

wherein R is Br if the backbone is synthesized through ATRP, H if synthesized through FRP, and a RAFT moiety if synthesized through RAFT.

In a still further aspect, the monomer has a structure:

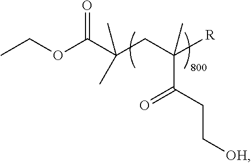

wherein R is Br if the backbone is synthesized through ATRP, H if synthesized through FRP, and a RAFT moiety if synthesized through RAFT.

In a further aspect, the monomer has grafted through a cyclic ester. In a still further aspect, the polymer has grafted through a cyclic ester. In a still further aspect, the cyclic ester is caprolactone. In yet a further aspect, the cyclic ester has a structure:

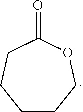

In a further aspect, a cyclic ester has been grafted through from a functionality on the monomer. In a still further aspect, a cyclic ester has been grafted from a functionality on the polymer.

In a further aspect, the monomer has a structure:

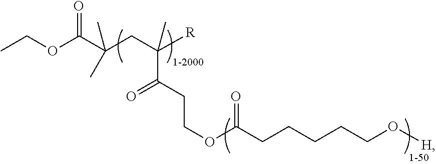

wherein R is Br if the backbone is synthesized through ATRP, H if synthesized through FRP, and a RAFT moiety if synthesized through RAFT.

In a still further aspect, the monomer has a structure:

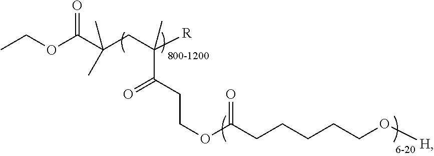

wherein R is Br if the backbone is synthesized through ATRP, H if synthesized through FRP, and a RAFT moiety if synthesized through RAFT.

In yet a further aspect, the monomer has a structure:

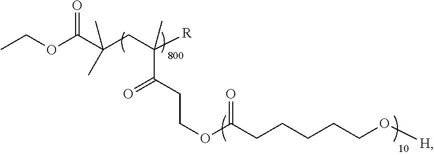

wherein R is Br if the backbone is synthesized through ATRP, H if synthesized through FRP, and a RAFT moiety if synthesized through RAFT.

In a further aspect, the polymer has a structure:

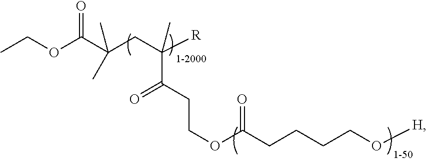

wherein R is Br if the backbone is synthesized through ATRP, H if synthesized through FRP, and a RAFT moiety if synthesized through RAFT.

In a still further aspect, the polymer has a structure:

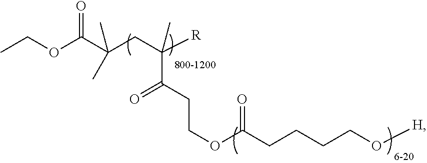

wherein R is Br if the backbone is synthesized through ATRP, H if synthesized through FRP, and a RAFT moiety if synthesized through RAFT.

In yet a further aspect, the polymer has a structure:

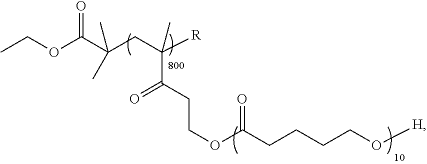

wherein R is Br if the backbone is synthesized through ATRP, H if synthesized through FRP, and a RAFT moiety if synthesized through RAFT.

In a further aspect, the monomer has been modified with a first binding functionality configured to react with any one of the third binding functionalities. Examples of first binding functionalities include, but are not limited to, hydroxyl moieties, amino moieties, carboxylic acid moieties, amide moieties, urea moieties, and furan moieties. In a still further aspect, the first binding functionality is selected from hydroxyl, amine, and furan. In yet a further aspect, the first binding functionality is a hydroxyl. In yet a further aspect, the first binding functionality is a furan. Examples of third binding functionalities include, but are not limited to, hydroxyl moieties, amino moieties, carboxylic acid moieties, amide moieties, urea moieties, and maleimide moieties. In a still further aspect, the first binding functionality is selected from hydroxyl, amine, and maleimide. In yet a further aspect, the first binding functionality is a maleimide.

In a further aspect, the monomer has a structure:

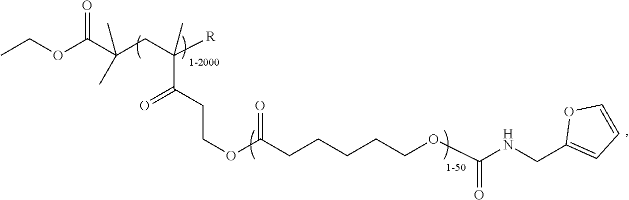

wherein R is Br if the backbone is synthesized through ATRP, H if synthesized through FRP, and a RAFT moiety if synthesized through RAFT.

In a still further aspect, the monomer has a structure:

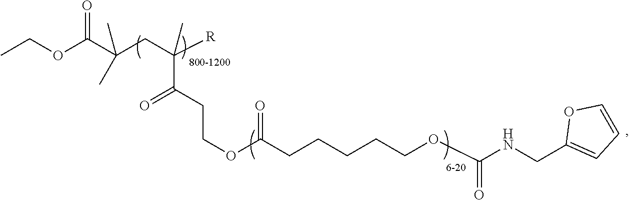

wherein R is Br if the backbone is synthesized through ATRP, H if synthesized through FRP, and a RAFT moiety if synthesized through RAFT.

In yet a further aspect, the monomer has a structure:

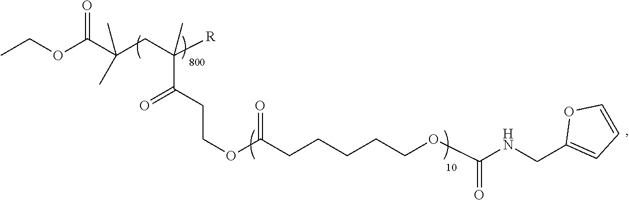

wherein R is Br if the backbone is synthesized through ATRP, H if synthesized through FRP, and a RAFT moiety if synthesized through RAFT.

In a further aspect, the monomer has a structure:

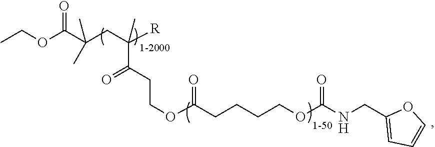

wherein R is Br if the backbone is synthesized through ATRP, H if synthesized through FRP, and a RAFT moiety if synthesized through RAFT.

In a still further aspect, the monomer has a structure:

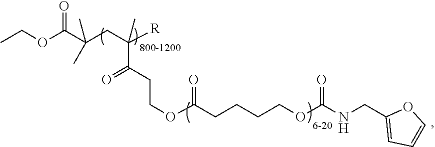

wherein R is Br if the backbone is synthesized through ATRP, H if synthesized through FRP, and a RAFT moiety if synthesized through RAFT.

In yet a further aspect, the monomer has a structure:

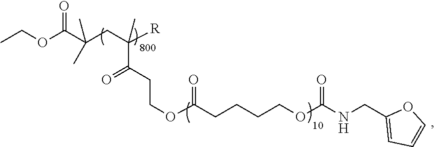

wherein R is Br if the backbone is synthesized through ATRP, H if synthesized through FRP, and a RAFT moiety if synthesized through RAFT.

In a further aspect, the monomer is reacted with the reversible cross-linker having a second binding functionality. Examples of second binding functionalities include, but are not limited to, hydroxyl moieties, amino moieties, carboxylic acid moieties, amide moieties, urea moieties, and furan moieties. In a still further aspect, the reversible cross-linker is a ureidopyrimidinone. In yet a further aspect, the reversible cross-linker has a structure:

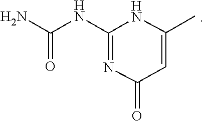

In a further aspect, the monomer is reacted with the reversible cross-linker having a pair of third binding functionalities. Examples of third binding functionalities include, but are not limited to, hydroxyl moieties, amino moieties, carboxylic acid moieties, amide moieties, urea moieties, and furan moieties. In a still further aspect, the reversible cross-linker is dimaleimide. In yet a further aspect, the reversible cross-linker has a structure:

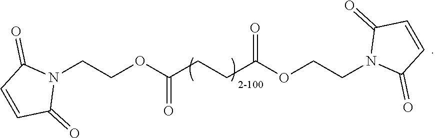

In an even further aspect, the reversible cross-linker has a structure:

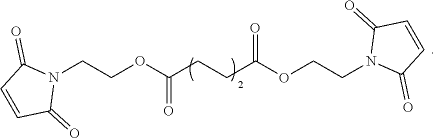

In a further aspect, the monomer is reacted with reversible cross-linker having a pair of third binding functionalities. In a still further aspect, each of the third binding functionalities is reacted with a first binding functionality.

### D. Incorporation of a Polymer Network into a Medical Device

In one aspect, the disclosed polymer network has been incorporated as a medical device. Examples of medical devices include, but are not limited to, implants, microneedle arrays, wound dressing pads, catheters, and drug delivery devices. In a further aspect, the polymer network is coated onto the device.

The present invention is primarily concerned with the treatment of human subjects, but the invention may also be carried out on animal subjects, particularly mammalian subjects such as mice, rats, dogs, cats, livestock and horses for veterinary purposes, and for drug screening and drug development purposes. Subjects may be of any age, including infant, juvenile, adolescent, adult, and geriatric subjects.

In various aspects, the medical device is a drug delivery device. Examples of drug delivery devices include, but are not limited, to implants and microneedle arrays. In a further aspect, the polymer network is coated onto the device.

In a further aspect, a therapeutic agent has also been incorporated into the drug delivery device. In a further aspect, the therapeutic agent comprises a protein therapeutic or a small molecule therapeutic. In yet a further aspect, the therapeutic agent is coated onto or dispersed in the device. In an even further aspect, the therapeutic agent is coated onto the device. In a still further aspect, the therapeutic agent is dispersed in the device.

In a further aspect, the therapeutic agent is released from the device. The release of the therapeutic agent may occur upon insertion or over a period of time. For example, in various aspects, the therapeutic agent may be released from the device over a period of about 1 minute to about 6 months. In a further aspect, the therapeutic agent may be released from the device over a period of about 1 minute to about 3 months. In a still further aspect, the therapeutic agent may be released from the device over a period of about 1 minute to about 1 month. In yet a further aspect, the therapeutic agent may be released from the device over a period of about 1 minute to about 2 weeks. In an even further aspect, the therapeutic agent may be released from the device over a period of about 1 minute to about 1 week. In a still further aspect, the therapeutic agent may be released from the device over a period of about 1 minute to about 3 days. In yet a further aspect, the therapeutic agent may be released from the device over a period of about 1 minute to about 1 day. In an even further aspect, the therapeutic agent may be released from the device over a period of about 1 minute to about 12 hours. In a still further aspect, the therapeutic agent may be released from the device over a period of about 1 minute to about 6 hours. In yet a further aspect, the therapeutic agent may be released from the device over a period of about 1 minute to about 1 hour. In an even further aspect, the therapeutic agent may be released from the device over a period of about 1 minute to about 30 minutes. In a still further aspect, the therapeutic agent may be released from the device over a period of about 30 minutes to about 6 months. In yet a further aspect, the therapeutic agent may be released from the device over a period of about 1 hour to about 6 months. In an even further aspect, the therapeutic agent may be released from the device over a period of about 6 hours to about 6 months. In a still further aspect, the therapeutic agent may be released from the device over a period of about 12 hours to about 6 months. In yet a further aspect, the therapeutic agent may be released from the device over a period of about 1 day to about 6 months. In an even further aspect, the therapeutic agent may be released from the device over a period of about 3 days to about 6 months. In a still further aspect, the therapeutic agent may be released from the device over a period of about 1 week to about 6 months. In yet a further aspect, the therapeutic agent may be released from the device over a period of about 2 weeks to about 6 months. In an even further aspect, the therapeutic agent may be released from the device over a period of about 1 month to about 6 months. In a still further aspect, the therapeutic agent may be released from the device over a period of about 3 months to about 6 months.

In a further aspect, the therapeutic agent may be released from the device over a period of less than about 1 minute. In a still further aspect, the therapeutic agent may be released from the device over a period of about 1 second to about 1 minute. In yet a further aspect, the therapeutic agent may be released from the device over a period of about 1 second to about 30 seconds. In an even further aspect, the therapeutic agent may be released from the device over a period of about 1 second to about 10 seconds. In a still further aspect, the therapeutic agent may be released from the device over a period of about 10 seconds to about 1 minute. In yet a further aspect, the therapeutic agent may be released from the device over a period of about 30 seconds to about 1 minute.

In a further aspect, the therapeutic agent is released without external stimulation.

In a further aspect, the therapeutic agent has a finite release rate. In a still further aspect, the therapeutic agent has a variable release rate.

In a further aspect, the therapeutic agent is not released at a temperature of less than about 75° F. In a still further aspect, the therapeutic agent has a release rate of 0 at a temperature of less than about 75° F. In yet a further aspect, the therapeutic agent has a release rate at a temperature of greater than about 90° F. that is greater than the release rate at a temperature of less than about 75° F. For example, the release rate at a temperature of greater than about 90° F. can be about two times, three times, four times, five times, six times, seven times, eight times, nine times, or ten times greater than the release rate at a temperature of greater than about 90° F.

### E. Examples

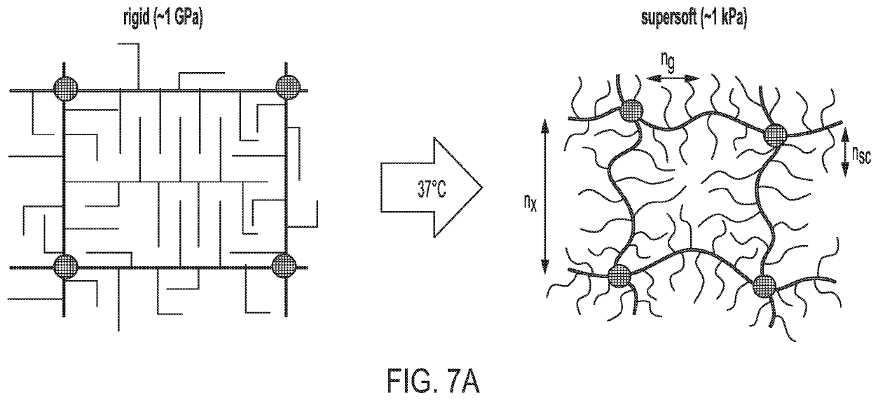

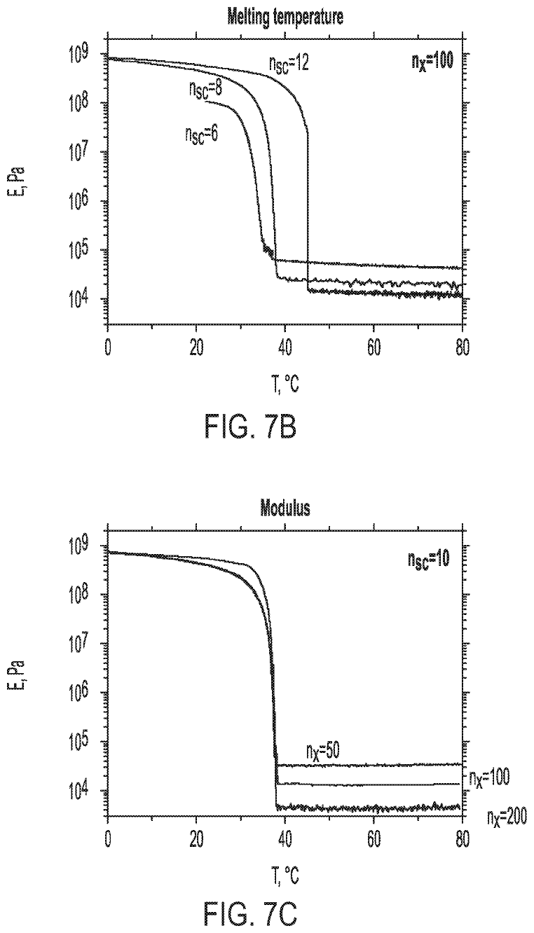

The disclosed bottlebrush elastomers have crystallizable side-chains that undergo hard-to-soft transition with architecturally programmable transition temperature and softness (FIG. 7A). Under ambient conditions (e.g., outside a human body), these materials are crystalline and hard as a PVC pipe, which allows easy handling, storage, and implantation. Once in the body, however, the side chains melt and the implant material becomes as soft as the surrounding tissue, minimizing inflammatory responses. A unique feature of this technology is dual architectural control over thermomechanical properties. Specifically, independent control over side chain length (n_{sc}) and crosslink density (nₓ) allows independent tuning of the transition temperature (FIG. 7B) and the Young's modulus (FIG. 7C) to match targeted implant destinations.

Referring to FIG. 7A, the melting of brush-like elastomers with crystallizable side chains demonstrates a transition from ˜1 GPa to ˜1 kPa stiffness in the physiological temperature range. Without wishing to be bound by theory, synthetic control over the brush architecture allows independent tuning of the transition temperature (FIG. 7B) and the Young's modulus (FIG. 7C).

Many implant destinations in the human body, such as brain, muscle, and adipose tissue, have a modulus of 10² to 10⁴ Pa, which is significantly below the entanglement limit of linear polymer melts (i.e., 10⁵ Pa). When a polymeric (10⁶-10⁹ Pa) or a metallic (>10⁹ Pa) implant is inserted, it creates a mechanical stress at the tissue-implant interface, aggravating inflammatory response, discomfort, and irritation. A universal strategy to synthesize bottlebrush polymers with mechanical properties exactly matching given tissue has been previously described. By controlling the degrees of polymerization of the side chains (n_{sc}), of the spacer between neighboring side chains (n_{g}), and of the strand backbone (nₓ), matching not only the exact modulus of the tissue, but also its strain stiffening behavior, was demonstrated. Bottlebrush polymers are inherently soft due to their extended backbone, thus eliminating chain entanglement. Therefore, they have the same modulus as a soft tissue (i.e., about 10³ to about 10⁵ Pa) without drawbacks such as leaking or drying. Because such softness is largely controlled by the network architecture (and not by the chemical composition), it is possible to incorporate other functions (e.g., adhesion and drug encapsulation) while retaining the same tissue-like softness.

Aliphatic polyesters such as poly(lactic acid) (PLA), poly(lactic-co-glycolic acid) (PLGA), and poly(caprolactone) (PCL) are the most common candidates for implants with great biocompatibility (Le Devedec et al. (2018) Mol. Pharm. doi:10.1021/acs.molpharmaceut.7b01102). PCL, with its great permeability and low degradation rate, has received much attention due to implant devices such as Capronor, a PCL-based contraceptive implant that demonstrated great phase II results. Poly(valerolactone) (PVL) is mechanically and chemically very similar to PCL, with great biocompatibility but lower Tₘ and T_{g}. However, PVL has a unique melting temperature range of 30-41° C. in bottlebrush compositions, making it particularly suitable for in vivo application.

Herein, tissue-adaptive, bio-compatible, and leakage-free bottlebrush elastomers useful in, for example, the design of biomedical devices including microneedle patches for drug delivery is described. Characterization reveals that in ex vivo conditions under room temperature these novel materials are hard (modulus >10⁸ Pa) and readily penetrate by themselves into a soft tissue (e.g., skin, brain, liver). In in vivo temperatures, the polymers are inherently soft (i.e., 10³-10⁵ Pa), matching that of surrounding tissue. Without wishing to be bound by theory, a drop of five orders of magnitude for the Young's modulus was achieved for these hard-to-soft bottlebrush elastomers, which significantly exceeds the conventional drop by a factor of 10²-10³× demonstrated by commodity plastics (see, e.g., FIG. 3A) and various biomaterials (see, e.g., FIG. 3B). Thermal and mechanical properties of these brush-like networks are controlled by n_{sc} and nₓ, capable of matching a specific tissue modulus and temperature, allowing custom-made solutions to specific individuals or scenarios. Additionally, controlled release behavior is shown, where the implant can release small molecules under only in vivo conditions.

The following examples are put forth so as to provide those of ordinary skill in the art with a complete disclosure and description of how the devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

1. Materials

δ-valerolactone (>98%) was obtained from Sigma-Aldrich and filtered to remove polymers. Benzene alcohol (PhOH), Tin(II) 2-ethylhexanoate(Sn(Oct)2>95%), ethylene glycol anhydrous, 4-Dimethylaminopyridine (DMAP>99%), Methacrylate chloride, Methacrylate anhydride, phenylbis(2,4,6-trimethylbenzoyl)phosphineoxide (BAPOs), tetrahydrofuran (THF), toluene anhydrous, ethanol anhydrous, Dicholomethane (DCM), Triethylamine(TEA) were purchased from Aldrich and used as received, as were all other reagents and solvents.

2. Grafting Through Synthesis of Polycaprolactone (PCL) Elastomers

a. Synthesis of Monomer (1a)

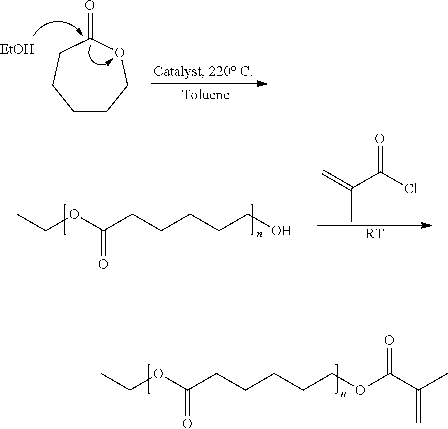

Dried ethanol (2.0 g, 44 mmol), ε-caprolactone (60 g, 526 mmol), 100 mL anhydrous toluene was added in an oven-dried flask. To the solution was added 3 Å molecular sieves and the mixture was dried for 48 h. The solution was filtered into a 200 mL round bottom flask. Dibutyltin dilaurate (100 mg) in 1 mL of toluene was added via syringe. The reaction mixture was heated to reflux and aliquots were removed periodically and analyzed by H-NMR. Between 6 and 8 h, the reaction became viscous and magnetic stirring became hard. After reaching to the degree of polymerization equal 10, the reaction was cooled to room temperature. The contents were then poured into methanol chilled in an ice bath to precipitate the polymer. The precipitation procedure was repeated two more times and the polymer filtered, washed with methanol, air dried, and then further dried under a vacuum.

The polymer (10 g, 8.8 mmol) dissolve in 100 mL DCM and dried with anhydrous MgSO₄ overnight. The polymer solution was filtered and transferred to a 200 mL oven-dried flask. Triethylamine (1 mL) was added to the flask and the temperature of mixture decrease to 5° C. using ice bath and methacryloyl chloride (1.1 g) in 10 mL anhydrous DCM was added dropwise to the mixture. The ice bath was removed and the temperature was increased to room temperature. The reaction was continued overnight. The mixture was filtered and filtrates were washed with water 3×200 mL. The contents were then concentrated and poured into methanol chilled in an ice bath to precipitate the polymer. The precipitation procedure was repeated two more times and the polymer filtered, washed with methanol, air dried, and then further dried under a vacuum.

b. Synthesis of Crosslinker (2a)

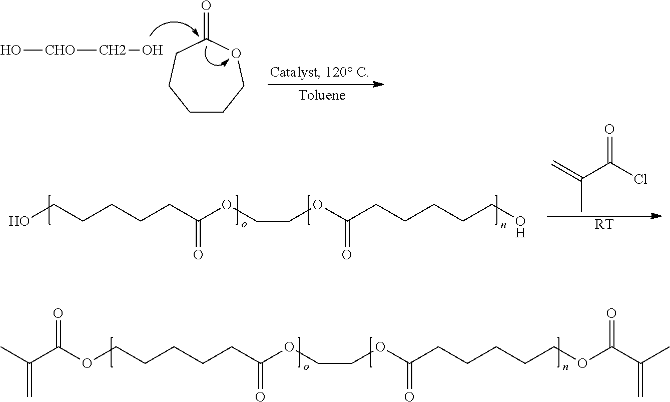

Dried ethylene glycol (1.0 g, 16 mmol), ε-caprolactone (46 g, 404 mmol), and 50 mL anhydrous toluene were added ton an oven-dried flask. To the solution was added 3 Å molecular sieves and the mixture was dried for 48 h. The solution was filtered into a 200 mL round bottom flask. Dibutyltin dilaurate (100 mg) in 1 mL of toluene was added via syringe. The reaction mixture was heated to reflux and aliquots were removed periodically and analyzed by ¹H NMR. Between 6 and 8 h, the reaction became viscous and magnetic stirring became hard. After reaching to the degree of polymerization equal 25, the reaction was cooled to room temperature. The contents were then poured into methanol chilled in an ice bath to precipitate the polymer. The precipitation procedure was repeated two more times and the polymer was filtered, washed with methanol, air dried, and then further dried under a vacuum.

The polymer (10 g, 8.8 mmol) was dissolved in 100 mL DCM and dried with anhydrous MgSO₄ overnight. The polymer solution was filtered and transferred to a 200 mL oven-dried flask. Triethylamine (1 mL) was added to the flask and the temperature of mixture was decreased to 5° C. using an ice bath before methacryloyl chloride (1.0 g) in 10 mL anhydrous DCM was added dropwise to the mixture. The ice bath was removed and the temperature increased to room temperature. The reaction was continued for overnight. The mixture was filtered and filtrates were washed with water 3×200 mL. The contents were then concentrated and poured into methanol chilled in an ice bath to precipitate the polymer. The precipitation procedure was repeated two more times and the polymer filtered, washed with methanol, air dried, and then further dried under a vacuum.

### C. Bottlebrush PCL Elastomer Films

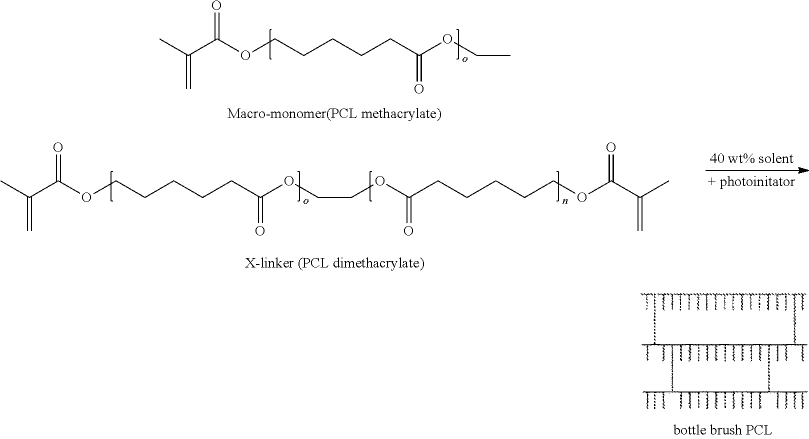

All bottlebrush elastomers were prepared by one-step polymerization of monomer (1a) (1140 g/mol) with different molar ratios of cross-linker (2a). The initial reaction mixtures contained: 60 wt % macro-monomers (1a), 1.5 wt % BAPOs photoinitiator, and 40.0 wt % PX as solvent. First, the mixtures were degassed by nitrogen bubbling for 30 minutes. Then, to prepare films, the mixtures were injected between two glass plates with a 2.3 mm PDMS spacer and polymerized at room temperature for 12 hrs under N₂ using a UV cross-linking chamber (365 nm UV lamp, 0.1 mW/cm⁻², 10 cm distance). Films were washed with chloroform (2× with enough to immerse and fully swell the films, each time for 8 hrs) in glass Petri dishes. The samples were then de-swelled with ethanol and dried in a 50° C. oven. The conversion of monomers to elastomers (gel fraction) was between 87 wt % to 95 wt % in every case.

3. Grafting Through Synthesis of Polyvalerolactone (PVL) Elastomers

Hard-to-soft bottlebrush polymers were synthesized with the grafting-through method. Specifically, PVL monofunctional macromonomers and difunctional crosslinkers were synthesized via ring-opening polymerization (ROP). Afterwards, the macromonomers and crosslinkers were polymerized by free radical polymerization initiated by a photo initiator. This synthesis route provides great control over side chain length (n_{sc}) and dispersity over grafting-from, in which side chain growth is sterically hindered. Desired over grafting-to, it also guarantees a 1.0 graft density since all units of the backbone are grafted. Without wishing to be bound by theory, control over these factors allows for the mechanical property of the bottlebrush network to be precisely defined, therefore providing ideal mechanical alleviation as implants.

a. Synthesis of Macromonomer, DP=10 (1b)

Dried Benzene alcohol (5.4 g, 50 mmol), δ-valerolactone (50 g, 500 mmol), 50 mL anhydrous toluene was added in an oven-dried flask. To the solution was added 3 Å molecular sieves and the mixture was dried for 48 h. The solution was filtered into a 200 mL round bottom flask. Tin(II) 2-ethylhexanoate (1 g) was added via syringe. The reaction mixture was heated to 110° C. and aliquots were removed periodically and analyzed by ¹H NMR. Between 6 and 8 h, the reaction became viscous and magnetic stirring became hard. After reaching to the degree of polymerization equal 10, the reaction was cooled to room temperature. The contents were then poured into hexane to precipitate the polymer. The polymer precipitate was filtered, washed with methanol, air dried, and then further dried under reduced pressure.

The polymer (55 g, ˜50 mmol) was dissolved in 100 mL DCM and dried with anhydrous MgSO₄ overnight. The solution was filtered and transferred to a 200 mL oven-dried flask. TEA (5.5 g) was added to the flask and the temperature of the mixture was decreased to 5° C. using an ice bath. Methacrylate chloride (5 g, 55 mmol) was added dropwise to the mixture. The ice bath was removed and the temperature was increased to 40° C. The reaction was allowed to continue overnight. The contents were then concentrated and poured into hexane chilled in an ice bath in order to precipitate the polymer. The polymer precipitate was filtered, washed with methanol, air dried, and then further dried under reduced pressure.

¹H NMR (CDCl₃): δ 1.91 (3H, —C(CH3)=CH2); 6.06, 5.55 (H, H, —C(CH3)=CH2); 2.31 (2 nH, —COOCH2-, PVL); 4.0 (2 nH, —CH2OOC—, PVL); 3.55, 4.02 (2H, 2H —CH2CH2-OCH3); 3.34 (3H, —CH2CH2-OCH3); MW=1100 from NMR; PDI=1.16 from GPC.

b. Synthesis of Crosslinker, DP=40 (2b)

Dried ethylene glycol (1.0 g, 16 mmol), δ-valerolactone (64 g, 640 mmol), 50 mL anhydrous toluene was added in an oven-dried flask. To the solution was added 3 Å molecular sieves and the mixture was dried for 48 h. The solution was filtered into a 200 mL round bottom flask. Tin(II) 2-ethylhexanoate (1 g) was added via syringe. The reaction mixture was heated to 110° C. and aliquots were removed periodically and analyzed by ¹H NMR. After 12 h, the reaction was complete, with ˜95% conversion and a degree of polymerization of 40. The reaction was cooled to room temperature. The contents were then poured into hexane chilled in an ice bath in order to precipitate the polymer. The polymer precipitate was filtered, washed with methanol, air dried, and then further dried under reduced pressure.

The polymer (64 g, ˜16 mmol) was dissolved in 100 mL DCM and dried with anhydrous MgSO₄ overnight. The solution was filtered and transferred to a 200 mL oven-dried flask. DMAP (2 g) was added to the flask and the temperature of the mixture was decreased to 5° C. using an ice bath. Methacrylate anhydride (3.7 g, 24 mmol) was added dropwise to the mixture. The ice bath was removed and the temperature was increased to 40° C. The reaction was allowed to continue overnight. The contents were then concentrated and poured into hexane chilled in an ice bath in order to precipitate the polymer. The polymer precipitate was filtered, washed with methanol, air dried, and then further dried under reduced pressure.

¹H NMR (CDCl₃): δ 1.91 (6H, —C(CH3)=CH2); 6.06, 5.55 (2H, 2H, —C(CH3)=CH2); 2.31 (2 nH, —COOCH2-, PVL); 4.0 (2 nH, —CH2OOC—, PVL); 4.28 (4H, —OCH2CH2O—); MW=4200 from NMR; PDI=1.26 from GPC.

c. Bottlebrush PVL Elastomer Films

All bottlebrush elastomers were prepared by one-step polymerization of macromonomer 1b (1108 g/mol) with different molar ratios of cross-linker (2b). The initial reaction mixtures contained: 60 wt % macro-monomers (1b), 1.5 wt % BAPOs photoinitiator, and 40.0 wt % toluene as solvent. First, the mixtures were degassed by bubbling dry N₂ for 30 minutes. Then, to prepare films, the mixtures were injected between two glass plates with a 2.3 mm PDMS spacer and polymerized at room temperature for 12 hrs under N₂ using a UV cross-linking chamber (365 nm UV lamp, 0.1 mW/cm⁻², 10 cm distance). Films were washed with THF (2× with enough to immerse and fully swell the films, each time for 8 h) in glass Petri dishes. The samples were then de-swelled with ethanol and dried in a 75° C. oven. The conversion of monomers to elastomers (gel fraction) was between 85 to 98 wt % depending on crosslinker concentration. Before tests, all samples were annealed at room temperature for 72 h to maximize crystallinity.

4. Grafting Through Synthesis of Polycaprolactone Bottlebrushes (DP=800)

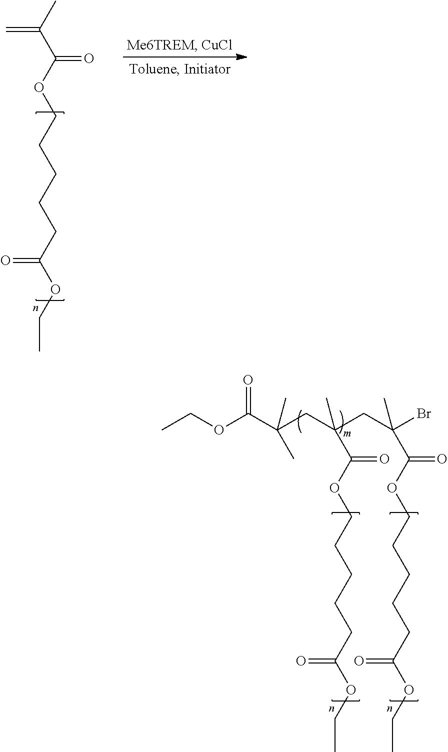

A 25 mL Schlenk flask equipped with a stir bar was charged with EBiB (1.9 mg, 10.0 μmol), pCL monomer (11.4 g, 10.0 mmol), Me₆TREN (10 μmol), and toluene (10.0 mL). The solution was bubbled with dry nitrogen for 1 hr, then CuCl (0.99 mg, 0.010 mmol) was quickly added to the reaction mixture under nitrogen atmosphere. The flask was sealed, back-filled with nitrogen, purged for 5 minutes, and then immersed in an oil bath thermostated at 45° C. The polymerization was stopped after 10 h at 80% monomer conversion, resulting in a bottlebrush pCL polymer with degree of polymerization (DP) of the backbone (n_{bb}) ˜800. The polymer was precipitated three times from methanol to purify, and dried under vacuum at room temperature until a constant mass was reached.

5. PHEMA Synthesis

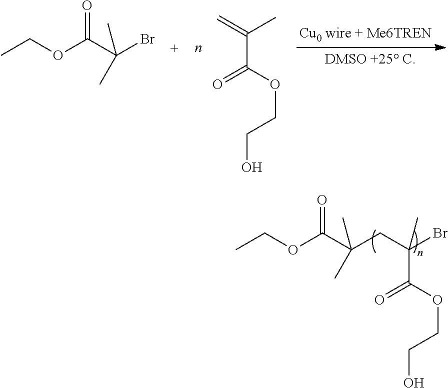

In a 25 mL air free flask, the reagents were added in the following orders under gentle stirring: ethyl bromoisobutyrate (1.95 mg, 0.01 mmol), monomer (HEMA, 1.95 mL, 12 mmol), ligand (Me6-TREN, 0.23 mg, 0.001 mmol) and solvent (DMSO, 4 mL). When the polymerization was performed at this high degree of polymerization a stock solution of ethyl bromoisobutyrate and Me6-TREN in DMSO was prepared. To reduce the viscosity of the polymer with high molecular weight, a DMSO/HEMA volume ratio of 2 was used. The mixture was deoxygenated using seven freeze-pump-thaw cycles from a dry ice/acetone bath. After the last deoxygenation cycle, Cu (0) wire wrapped around a stirring bar was loaded into the reaction vessel under positive argon pressure, at t=0. The reaction vessel as placed in a water bath thermostatted at 25° C. with stirring. The side arm of the flask was purged with argon before it was opened for sampling at the predetermined times with an airtight syringe. At each time, a small amount of the sample was dissolved in d⁶-DMSO for the analysis of monomer conversion by ¹H-NMR, to measure the degree of polymerization. The polymerization was stopped by dilution of product with THF when the conversion was reached to 80% and the polymer purified by precipitation of polymer in ether and the rest was kept in a small vial for acetylation.

6. Grafting from Synthesis of Poly-Caprolactone Bottlebrushes from Hydroxyl Groups of PHEMA

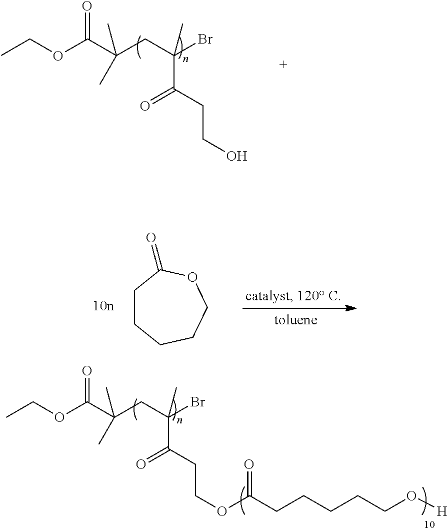

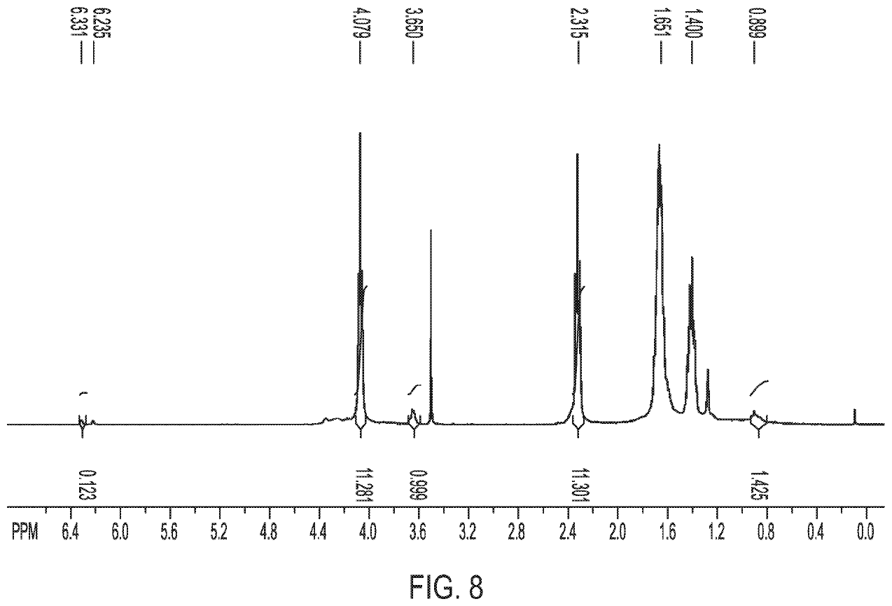

pHEMA (1.5 g, 14 μmol, 11.2 mmol hydroxyl groups) was dissolved in anhydrous DMF (50 mL) in a 100 mL oven dried flask. After complete dissolution, 15.0 g caprolactone and 0.1 g dibutyltin dilaurate were added to the flask and purged with nitrogen for 10 min and was placed in a 110° C. oil bath. The degree of polymerization was tracked by ¹H-NMR experiment (FIG. 8). When the degree of polymerization of caprolactone reached 10, the temperature of the reaction was decreased to room temperature and the polymer was precipitated in ether two times. ¹H NMR (CDCl₃): δ 1.04 0.88 (3H, —CH2CH3(COO—)—); 3.65 (2H, —CH2CH3(COO—)—), 2.31 (2H, —COOCH2-,PCL) 4.0 (2H, —CH2OOC—, PCL); MW=8.7×10⁵ from NMR; PDI=1.36 from GPC.

7. Functionalization of Hydroxyl End Groups (A)

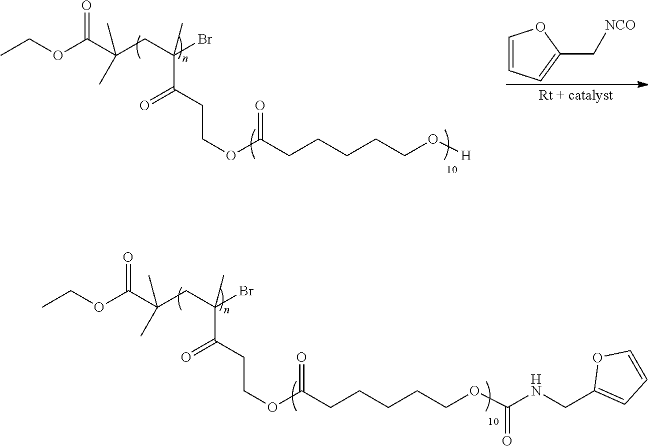

The polymer (pHEMA₈₀₀-g-pCL₁₀) (20 g, 21 μmol, 950,000 g/mol) was dissolved in 150 mL anhydrous DCM and oven-dried with anhydrous MgSO₄ overnight. The polymer solution was filtered and transferred to a 250 mL oven-dried flask. Dioctyltin dilaurate (100 mg) in 1 mL of anhydrous DCM was added via syringe. Then 0.15 g furan isocyanate was added drop-wise to the flask. The reaction mixture was stirred overnight and analyzed by ¹H-NMR to measure the average mole percentage of furan groups on the polymer chains. The contents were then concentrated and poured into methanol chilled in an ice bath to precipitate the polymer. The precipitation procedure was repeated two more times and the polymer filtered, washed with methanol, air dried, and then further dried under a vacuum. ¹H NMR (CDCl₃): δ 1.04 0.88 (3H, —CH2CH3(COO—)—); 3.65 (2H, —CH2CH3(COO—)—), 2.31 (2H, —COOCH2-,PCL) 4.0 (2H, —CH2OOC—, PCL); 6.33 (H, ═CHO, furan) 6.23 (H, CH═CHO, furan); 12% furan graft density from NMR.

8. Synthesis of Dimaleimide Cross-Linker (B)

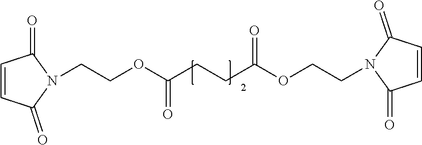

N-(2-Hydroxyethyl) maleimide (4.25 g, 0.03 mol) and 50 mL DCM were added to a 100 mL oven-dried flask. The temperature of the mixture was decreased via an ice bath and 10 mL triethylamine was added to the flask gently with stirring. After complete dissolution, 11.5 g adipoyl chloride was added drop-wise to the reaction over 30 minutes. The reaction temperature was increased to room temperature and the reaction was continued overnight. The mixture was filtered and the filtrate was washed with water 3×200 mL. The mixture was dried using anhydrous MgSO₄ overnight and then was filtered and the solvent evaporated to afford a white, solid, powder-shaped product.

9. Preparation of Dynamic PCL Network

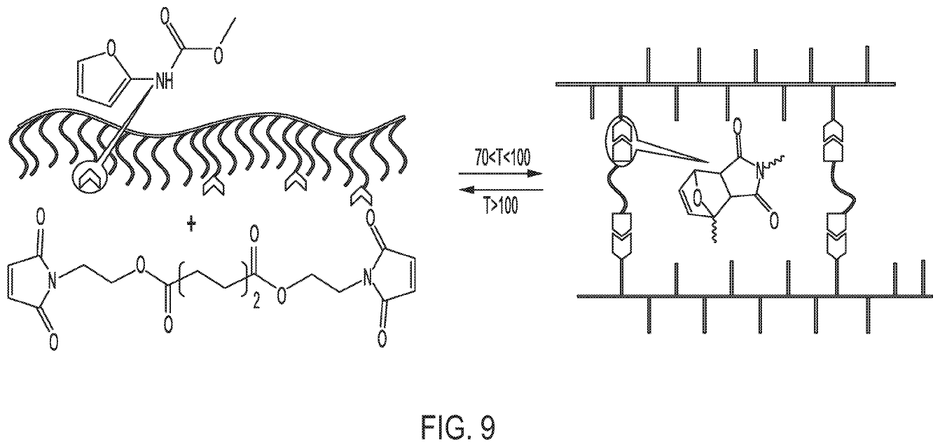

The mixture of product A, furan functionalized bottle brush (pHEMA₈₀₀-g-pCL₁₀)g-, and B, dimaleimide cross-linker, with different ratio between mole number of furan groups and maleimide groups were mixed in a mold and placed in a 70° C. oven. After 30 minutes, a network was obtained due to a Diels Alder reaction between the furan and the maleimide groups (FIG. 9). These Diels Alder bonds are reversible in temperature higher than 100° C. and provide self-healing and remoldable properties to the materials.

10. Temperature Dependence of Hard-to-Soft Elastomers

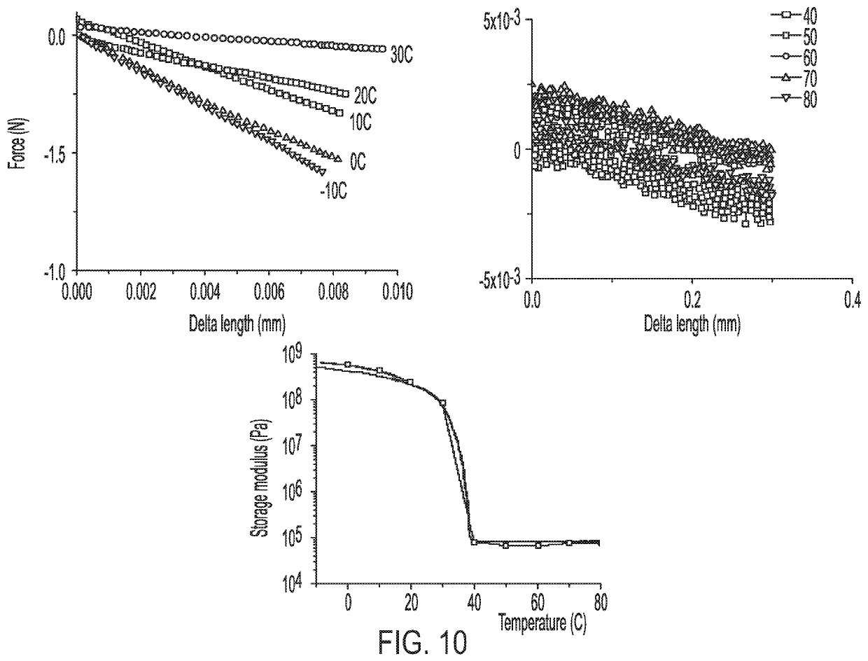

Dog bone-shaped samples with bridge dimensions of 12 mm×2 mm×1 mm were loaded into an RSA-G2 DMA (TA Instruments) and subjected to uniaxial oscillation under 1° C./min increasing temperature at a constant frequency of 2 Hz. From −10° C. to its transition temperature, which is determined by DSC (TA Instruments) in a typical heat/cool/heat run from −80° C. to 60° C., the strain used was 0.05%. Above transition temperature to 80° C., the strain used was 5%. Without wishing to be bound by theory, this increase is likely due to the worse signal to noise ratio generated at low strain for much lower modulus. The accuracy of this measurement is verified by measuring modulus individually per 10 degree Celsius (FIG. 10) after >20 min of resting at each temperature. Referring to FIG. 10, the match between individual tests and the sweep test suggests equilibrium throughout the test.

11. Thermal Property of Hard-to-Soft Bottlebrush

Thermal properties of bottlebrush polymers are analyzed by DSC and compared with their linear counterparts. For bottlebrush polymers, attached side chain have more restrain and lamellar are expected to be smaller than linear polymer with equivalent degree of polymerization. As shown in Table 1, compared to the macromonomer before crosslinking (n_{sc}=8 nₓ=0), crosslinked bottlebrush (n_{sc}=8, nₓ=50) has a Tm decrease from 38.1° C. to 33.3° C. Degree of crystallinity also decreased from 0.59 to 0.33, these proves smaller crystalline regions due to restricted chain conformation. The change in melting point is crucial to this work as it brings transition temperature of the polymer between room temperature and body temperature. This contrast allows the polymer to differentiate in vivo and ex vivo conditions.

Chain length effect on semi-crystalline behavior of linear aliphatic polyesters is well understood. The lamellar structure prevents very short chain (dimers or trimers) from forming crystalline phase, making the polymer amorphous. As the chain becomes longer (M_{w}>500), lamellar thickness increases, larger crystalline phase appear, and an increase of both melting entropy and melting point is typically observed. Beyond a certain limit, chain folding prevents the lamellar from further thickening and Tm stops changing. This is evident in DSC results of our pVL macromonomers. Similar theory can be applied to the effect of side chain length (n_{sc}) in poly-valerolactone bottlebrush polymers. As n_{sc} increases within range of interest (6-14) (Table 1), melting point and crystallinity increases in the range of 30˜42° C., hinting bigger crystalline domains. Since body temperature can vary within individual, this range of melting point allows us to shift transition temperature of the polymer, better serving peri-implant tissues with different temperature. The change in crystallinity is also evident in hard state modulus (FIG. 3). Larger crystalline domains changes the blend ratio between crystalline and amorphous domains, yielding harder modulus. For bottlebrush with n_{sc}=6 to 14, room temperature modulus change from 95 MPa to 0.44 GPa, all lower than that of linear PVL under similar conditions (>0.6 GPa).

Since thermal property of a polymer blend is typically dictated by weight content, the <1 wt % amount of crosslinkers plays negligible role in thermal properties of the network. As seen in Table 1, for n_{sc}=8, crystalline behavior does not significantly change when nₓ ranges from 50 to 400.

TABLE 1

| | | Tₘ | ΔHₘ | Tc | ΔH_{c} | |
|---|---|---|---|---|---|---|
| n_{sc} | nₓ | (° C.) | (J/g) | (° C.) | (J/g) | X_{c} |
| 6 | 50 | 29.3 | 43.9 | −13.5 | 33.3 | 0.23 |
| 8 | 0 | 38.1 | 85.9 | 18.4 | 85.0 | 0.59 |
| 8 | 50 | 33.3 | 47.9 | −9.9 | 48.2 | 0.33 |
| 8 | 100 | 32.4 | 45.9 | −10.9 | 43.0 | 0.3 |
| 8 | 200 | 31.3 | 46.8 | −10.8 | 42.6 | 0.3 |
| 8 | 400 | 32.1 | 45.0 | −4.4 | 43.5 | 0.3 |
| 10 | 50 | 35.2 | 55.6 | −0.2 | 54.8 | 0.38 |
| 12 | 50 | 42.0 | 49.1 | 8.3 | 45.9 | 0.32 |

n_{sc} is the degree of polymerization for side chain. nₓ is determined by composition the ratio of vinyl groups between macromonomer and crosslinker. Melting point (Tₘ), Enthalpy of melting (ΔHₘ), Crystallizing point (T_{c}), Enthalpy of crystallization (ΔH_{c}) data are measured by DSC analysis at 10 C./min. Degree of Crystallinity(X_{c}) is calculated with equation X_{c} = ΔHm/ΔHₘ⁰ where ΔHₘ⁰ is the melting enthalpy of 100% crystalline pVL which is 144 J/g.

12. Bottlebrush Softness of Hard-to-Soft Bottlebrush Polymers

For un-entangled polymer networks such as the disclosed hard-to-soft bottlebrush polymer in its soft state, it has been established that the structural modulus is characterized by the monomer units in between crosslinks, expressed as:

$$E = \frac{3 ρ {k}_{b} T}{{n}_{s}} \qquad ( 1 )$$

where E—Young's modulus, ρ—density of monomer units, k_{b}—Boltzmann constant, T—Kelvin temperature, and nₛ—monomer units between crosslinks. In the disclosed bottlebrush polymers, given every unit between crosslinks are macromonomers with n_{sc} units of valerolactone and 1 methacrylate backbone, this equation can be rewritten as

$$E = \frac{3 ρ {k}_{b} T}{{n}_{x} ( {n}_{sc} + 1 )} \qquad ( 2 )$$

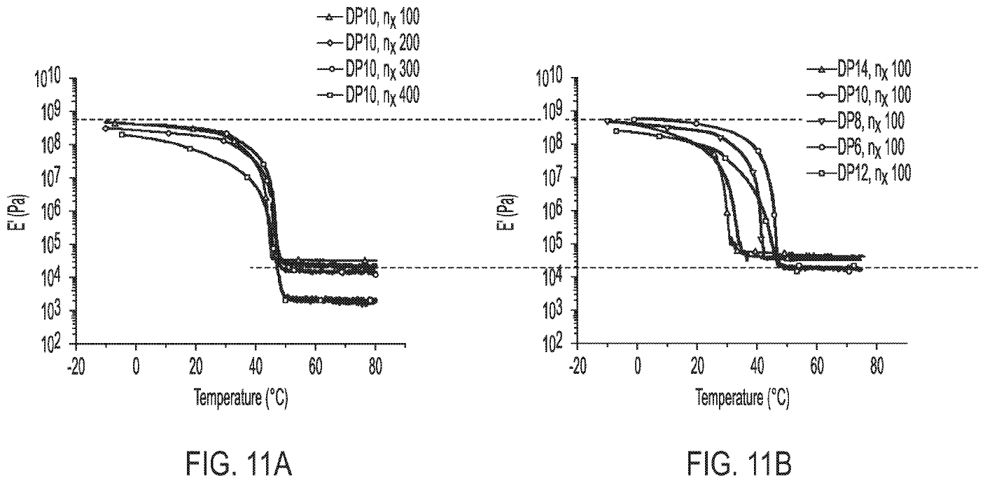

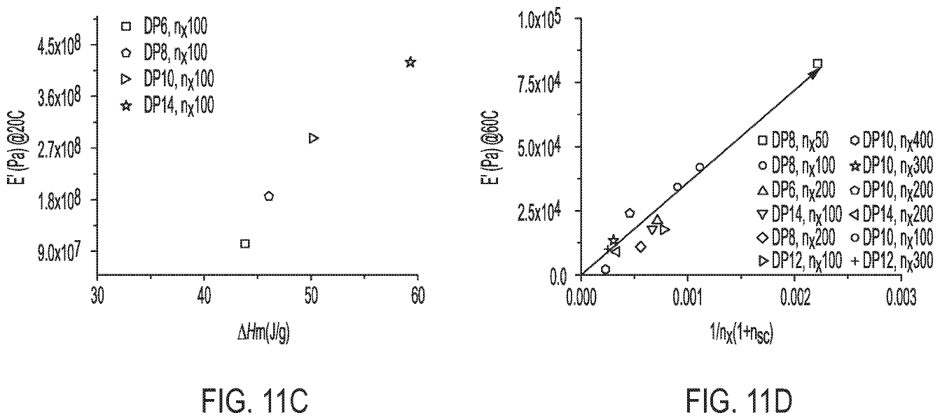

To display this relationship, the Young's modulus of series of samples with different n_{sc} and nₓ at 60° C. are obtained from their DMA analysis (E≈E′ for network polymers) and plotted against nₓ(n_{sc}+1) in FIG. 11A-D. For n_{sc}=6-14, nᵣ=50-100, a generally linear trend agrees with Eq. 2. Note that for very low crosslink density (nₓ>400), modulus fall slightly below the trend line. Without wishing to be bound by theory, this can be attributed to increasing free chain ends diluting the network when the backbone has less crosslinks per chain.

To individually access the effect of nₓ and n_{sc} on the overall thermal dependent mechanical properties throughout hard and soft state, two series of DMA analysis are compiled with control over each variable. With the same macromonomer with n_{sc}=10, the effect of nₓ ranging 100-400 is show in FIG. 11B. Consistent with DSC results, their crystallinity is independent of crosslinking density, yielding similar hard state modulus and transition temperature. The soft state modulus, on the other hand, decreases with increasing nₓ. It is worth noting that the modulus range matches the vast majority of soft tissue in the human body from stiffer muscle (˜10⁴ Pa) to very soft brain matter (˜10² Pa). Without wishing to be bound by theory, this allows for the softness of the disclosed polymer to be fine-tuned under in vivo conditions independently.

On the other hand, different macromonomers (n_{sc}) with the same crosslink density (nₓ) make more changes to the network. The effect is twofold, as seen in FIG. 11C. Increased side chain mass allows bigger crystalline domains to form, rising hard state modulus and melting temperature. On the other hand, since the longer side chain create more mass between crosslinks, ns increases, so the soft state becomes softer. Combined, longer side chain increases the modulus contrast between states, which is favored, but at the cost of increased melting temperature. This can be unfavored when the implant is intended for lower temperature organs such as epidermal implants. Typically, n_{sc}=10 is the upper limit for a 3 7° C. in silico condition. Note from eq. 2 that E˜nₓ(nₓ+1), as long as n_{sc} is changed accordingly to keep nₓ(n_{sc}+1) roughly constant, such as the two highlighted points in FIG. 7B and FIG. 7C (n_{sc}=6, nₓ=100 and n_{sc}=12, nₓ=200), one can still independently change the transition temperature despite this convoluted effect of Nsc on the network. Combining nₓ and n_{sc}, an oblique control of soft state, tissue specific modulus and transition temperature is achieved purely with chain formation, without changing its bulk chemical structure. The materials, therefore, have tremendous potential as universal implant solutions for tissues with various modulus and temperature, while expected to have exactly the same physiochemical property and biocompatibility.

Referring to FIG. 11A and FIG. 11B, the degrees of polymerization of the side chains (n_{sc}) and the network strands (nₓ) allows independent control over modulus in the soft state (FIG. 11A) and transition temperature from the hard state to the soft state (FIG. 11B). Network properties including hard state modulus, transition temperature, and soft state modulus are defined by nₓ and n_{sc}. When n_{sc} is decreased, three effects occur simultaneously: (i) hard state modulus; (ii) soft state modulus increases; and (iii) transition temperature increases.

Referring to FIG. 11C, in the hard state, melting enthalpy and transition temperature (SI) increases with n_{sc} indicating larger crystalline domains and higher crystallinity. This leads to an increase in modulus, as the percentage of hard phase increases in a semi-crystalline system. The <2% crosslinker can be neglected here.

Referring to FIG. 11D, in the soft state, the Young's modulus increases linearly with crosslink density as E˜1/nₓ(1+n_{sc}). As such, the hard-to-soft transition can be tuned to fit the stiffness of a targeted tissue at the body temperature by adjusting the n_{sc} and nₓ architectural parameters.

13. Release Behavior of Hard-to-Soft Bottlebrush Polymers

While having >90 wt % side chain units, hard-to-soft bottlebrush polymers keep the property of PVL as a great controlled drug release candidate with good loading efficiency, low cytotoxicity, and controllable long-term degradation. However, previous work with linear PVL can only be applied in its crystalline state, which hinders effective drug release unless additional enzyme is introduced. This is traditionally alleviated by copolymerization with heteromonomers like allyl valerolactone, decreasing the crystallinity. The disclosed bottlebrush polymer, on the other hand, in its soft state, has much more mobile chains and, therefore, a higher diffusion coefficient. The contrast of the diffusion in its crystalline hard phase and soft phase enables a thermally triggered drug release.

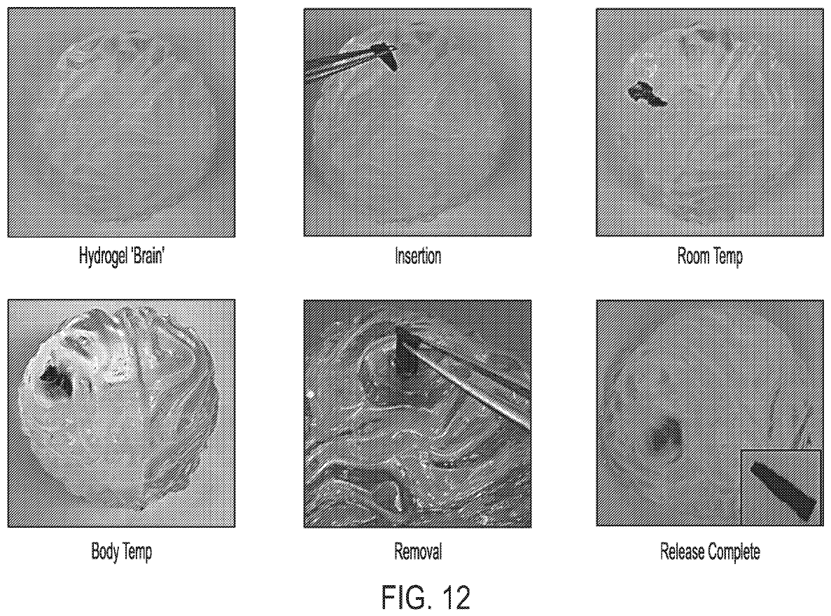

A simple in silico demonstration is illustrated in FIG. 12. After post-loading a dye (crystal violet), the “implant” is applied to a dummy hydrogel “brain.” The crystalline hardness allows self-penetration without a precut, while the softness under 37° C. poses minimal strain to its surrounding “tissues.” More importantly, the same loaded material, which has undergone 24 h of room temperature storage in water without visible leakage, shows great contrast upon temperature elevation in the “brain.” Throughout 45 minutes at room temperature, no visible release can be observed in the vicinity of the “implant.” After the temperature was raised to 37° C., within 5 minutes purple diffusion patterns became visible, which develops into a dark purple aura during the same 45 minute timespan. This release behavior has great potential as a drug delivery implant as it can monitor local temperature changes including inflammation, which is a common side effect of implants, and release its content accordingly.

Referring to FIG. 12, a dummy hydrogel “brain” was used to resemble in vivo tissue with ample water. An “implant” was made with Nx=50 Nsc=10 hard-to-soft elastomer with crystal violent as a visible small molecule simulating drugs desired to be released. When applied in room temperature, the “implant” was hard and could readily penetrate the polyimide hydrogel, leaving a clear “wound” cut. Within 45 minutes of room temperature storage, the “brain” remained clear and the “implant” can be removed and reinserted with ease, suggesting a great advantage in surgery handling. The “brain” with the “implant” was then kept at a raised temperature in a water bath for another 45 minutes, during which time the release was visible to the human eye, turning the vicinity of the “implant” into a purple tone. This is due to its thermal transition as the network becomes loose and mobile. After 45 minutes, the “implant” completed its transition and had a modulus similar to the surrounding hydrogel, so much so that it cannot be retracted from the “brain” because the “wound” is pressurizing and securing it. Without wishing to be bound by theory, this has great implication to eliminate relative movement between any implant and its surrounding tissue. The “implant” was then allowed to cool down to room temperature, at which time it could again be easily removed, leaving a purple mark at the “wound.”

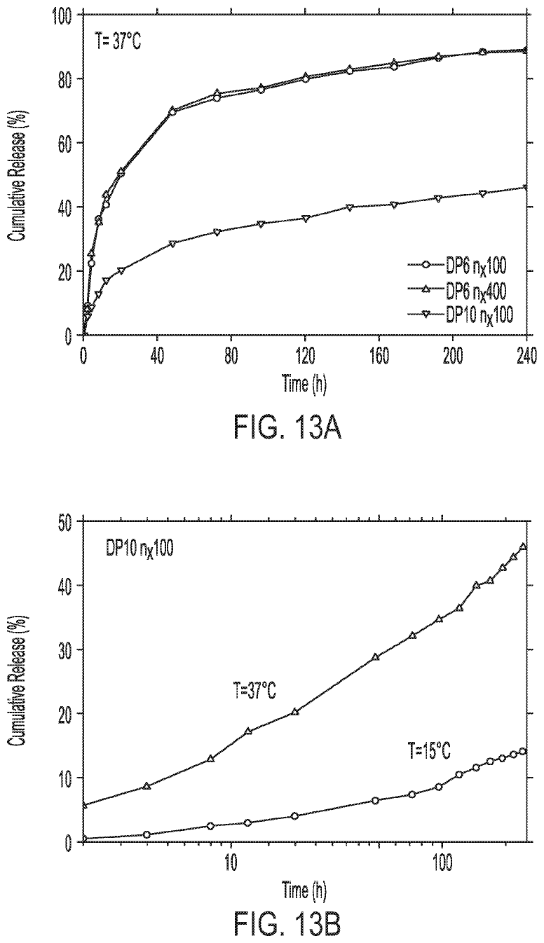

FIG. 13A and FIG. 13B show the effect of brush architecture and temperature on the release rate. Specifically, release profiles of rhodamine-B (0.1 wt %) from PCL networks as a function of the degree of polymerization of side chains (n_{sc}) (FIG. 13A) and temperature (FIG. 13B) are shown.

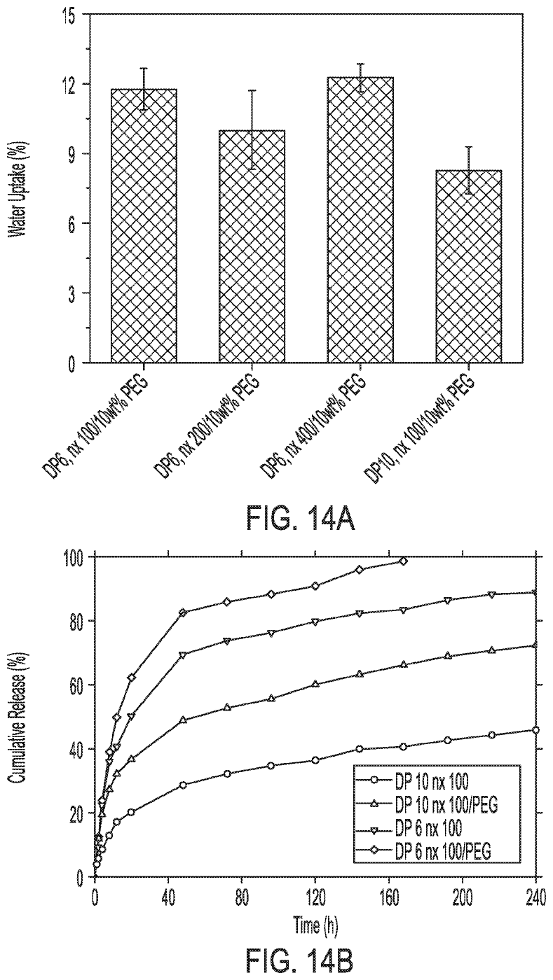

The release rate can be further enhanced by adding polyethylene glycol (PEG) side chains to PCL brush network strands that promote water uptake (FIG. 14A and FIG. 14B). For example, 10 wt % of PEG side chains results in ca. 10% of water uptake (FIG. 14A), which causes 20-30% increase of release rate of Rhodamine B (FIG. 14B).

Referring to FIG. 14A, PCL elastomers with different crosslink densities and the same weight fraction of PEG side chains (10 wt %) show similar water uptake of about 10%. Referring to FIG. 14B, release rate increases upon adding PEG side chains to the brush network strands.

In sum, the first biocompatible materials that are hard in ex vivo conditions but become as soft as its surrounding tissues (10³˜10⁵ Pa) in vivo are disclosed. Unprecedented modulus change (˜10⁵ times) is demonstrated within a narrow thermal interval around body temperature (30-42° C.). This material is shown to be hard for ex vivo handling and storage and soft to minimize adverse reaction caused by mechanical mismatch. A versatile strategy has been developed for different implant applications with various tissue softness and temperature by tuning chain formation, while retaining the same chemical composition and biocompatibility. The thermal transition also enables thermal-triggered drug delivery and potential advanced implant applications.

### F. Prophetic Examples

1. Dynamic Network Formation Based on Hydrogen Bonding

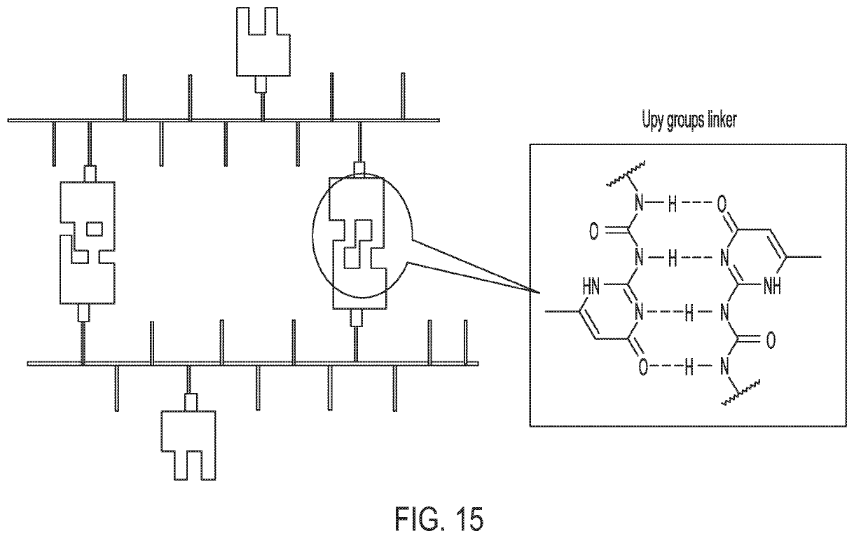

FIG. 15 shows an exemplary dynamic polymer network formed based on hydrogen bonding between ureidopyrimidone functional groups on the side chains of the polymer backbone.

The foregoing is illustrative of the present invention, and is not to be construed as limiting thereof. The invention is defined by the following claims, with equivalents of the claims to be included therein.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

1. A polymer network having an elastic modulus of at least about 10⁸ Pa at a temperature of less than about 75° F. and an elastic modulus of from about 10² Pa to about 10⁵ Pa at a temperature of greater than about 90° F.

2. The polymer network of claim 1, wherein the polymer network has an elastic modulus of at least about 10⁹ Pa at a temperature of less than about 75° F.

3. The polymer network of claim 1, wherein the polymer network has been incorporated into a medical device or as a coating for a medical device.

4. The polymer network of claim 3, wherein the medical device is an implant.

5. The polymer network of claim 3, wherein the medical device is a drug delivery device.

6. (canceled)

7. The polymer network of claim 1, wherein the polymer network comprises:
(a) at least two polymer backbones;
(b) a plurality of polymeric residues pendant from the polymer backbones, wherein the plurality of polymeric residues has a degree of polymerization of from about 1 to about 300, wherein the plurality of polymeric residues has a contour length of from about 1 nm to about 1 μm, wherein the plurality of polymeric residues has a softening transition temperature of from about −4° F. to about 140° F.; and
(c) optionally, a side chain moiety pendant from the polymer backbones, wherein the side chain moiety either has a first binding functionality or is bonded to a reversible cross-link moiety,
wherein the polymer network has a grafting density of from about 0.01 to about 1.

8. The polymer network of claim 7, wherein the polymer network further comprises one or more of:
(d) an irreversible cross-link moiety covalently bonded to the two polymer backbones;
(e) a plurality of reversible cross-link moieties having a first end and a second end, wherein each first end is covalently bonded to one of the two polymer backbones, and wherein each second end is bonded to each other; and
(f) a reversible cross-link moiety having a first end and a second end, wherein the first end is bonded to one side chain moiety, and wherein the second end is bonded to a different side chain moiety

9. The polymer network of claim 8, wherein the polymer network has a cross-linking density of from about 10⁻⁸ to about 10⁻³ mol/cm³.

10. The polymer network of claim 8, wherein the polymer network comprises the irreversible cross-link moiety.

11. The polymer network of claim 10, wherein covalently bonded is via the reaction of two functionalities selected from the group consisting of an amine residue, an alkyne residue, an azide residue, a hydroxyl residue, an aldehyde residue, an acrylate residue, a methacrylate residue, a vinyl residue, and a thiol residue.

12. The polymer network of claim 10, wherein covalently bonded is via the reaction of an amine residue, an aldehyde residue

13. The polymer network of claim 8, wherein the polymer network comprises the plurality of reversible cross-link moieties.

14. The polymer network of claim 13, wherein each reversible cross-link moiety is a ureidopyrimidinone residue, a maleimide residue, a catechol residue, a thiol residue, or a furfuryl residue.

15. The polymer network of claim 8, wherein the polymer network comprises the reversible cross-link moiety.

16. The polymer network of claim 15, wherein the reversible cross-link moiety is a dimaleimide residue, an aldehyde residue, an isocyanate residue, an alkyne residue, an alkene residue, or an azide residue.

17. The polymer network of claim 8, wherein one or more of the irreversible cross-link moiety, the plurality of reversible cross-link moieties, and the reversible cross-link moiety is biodegradable.

18. The polymer network of claim 7, wherein the polymer backbone is a polyester backbone, a polyacrylate backbone, or a methacrylate backbone.

19. The polymer network of claim 7, wherein the polymeric residue is a polyester residue, a polyacrylate residue, or a polymethacrylate residue.

20. The polymer network of claim 7, wherein the polymeric residue is a polyester residue selected from a polycaprolactone residue and a polyvalerolactone residue.

21. The polymer network of claim 1, wherein the polymer network comprises the reaction product of:
(a) a monomer selected from polyvalerolactone, polycarbonate, polycaprolactone methacrylate, polylactide methacrylate, polyglycolide methacrylate, polycaprolactone methacrylate, polycaprolactone acrylate, polylactide acrylate, polyethylene glycol, poly(2-ethyl-2-oxazoline), polyhydroxyalkanoate methacrylate, polyglycolide acrylate, and copolymers thereof; and
(b) one or more of:
(i) an irreversible cross-linker having two or more polymerizable functionalities selected from alkylene, alkene, acrylate, methacrylate, and epoxy;
(ii) a reversible cross-linker having a second binding functionality, wherein the second binding functionality on one reversible cross-linker can bond to the second binding functionality on a second reversible cross-linker; and
(iii) a reversible cross-linker having a pair of third binding functionalities;
wherein the polymer network has a grafting density of from about 0.01 to about 1; and
wherein the polymer network has a cross-linking density of from about 0.01 mole % to about 100 mole %.
